(19) United States
(12) Patent Application Publication
(10) Pub. No.: US 2025/0250195 A1
(43) Pub. Date: Aug. 7, 2025

(54) PLASMA ACTIVATED COATED SUBSTRATES

(71) Applicant: THE UNIVERSITY OF SYDNEY, New South Wales (AU)

(72) Inventors: Marcela BILEK, New South Wales (AU); Clara Thao Hoang TRAN, New South Wales (AU); Stuart FRASER, New South Wales (AU); Aaron GILMOUR, New South Wales (AU); Badwi BOUMELHEM, New South Wales (AU)

(21) Appl. No.: 18/857,721

(22) PCT Filed: Apr. 19, 2023
(86) PCT No.: PCT/AU2023/050317

§ 371 Date: Oct. 17, 2024

(30) Foreign Application Priority Data
Apr. 19, 2022 (AU) 2022901034

(51) Int. Cl. C03C 17/34; C12N 5/00

(52) U.S. Cl. CPC C03C 17/34; C12N 5/0068; C03C 2218/153; C03C 2218/31; C12N 2529/10; C12N 2533/12; C12N 2533/30; C12N 2533/52; C12N 2533/54; C12N 2537/10

(57) ABSTRACT

Substrates modified with plasma-activated coatings are provided, as are processes for their preparation. The coated substrates may possess high transparency and are radical rich, enabling covalent attachment of biomolecules, for example proteins.

### FIELD OF THE DISCLOSURE

The present disclosure relates to substrates modified with plasma-activated coatings and to processes for their preparation. The modified substrates may be treated with biomolecules to provide substrates having covalently attached biomolecules.

### BACKGROUND OF THE DISCLOSURE

Glass is a favourable substrate for high resolution and fluorescence imaging due to its low autofluorescence and high transparency. However, glass is an inhospitable substrate for in vitro cell culture. Most cells bind loosely to glass surfaces and can easily detach when the surrounding environment changes. Efforts to modify glass surfaces with a protein layer such as collagen, laminin or fibronectin have shown improvement in cell adhesion and proliferation (M. J. Cooke, S. R. Phillips, D. S. H. Shah, D. Athey, J. H. Lakey, S. A. Przyborski, Cytotechnology 2008, 56, 71). These extracellular matrix proteins can be attached on glass surfaces by physical adsorption; (E. Dejana, S. Colella, L. R. Languino, G. Balconi, G. C. Corbascio, P. C. Marchisio, J Cell Biol 1987, 104, 1403 and R. Bai, L. Tian, Y. Li, J. Zhang, Y. Wei, Z. Jin, Z. Liu, H. Liu, Stem Cells International 2019, 2019, 6708435) or by chemical linkers (M. C. Prewitz, F. P. Seib, M. von Bonin, J. Friedrichs, A. Stißel, C. Niehage, K. Müller, K. Anastassiadis, C. Waskow, B. Hoflack, M. Bornhäuser, C. Werner, Nature Methods 2013, 10, 788). However, physically adsorbed molecules have a high risk of being detached from the surface during cell culture (O. G. Alamdari, E. Seyedjafari, M. Soleimani, N. Ghaemi, Avicenna J Med Biotechnol 2013, 5, 234) and thus, covalent attachment is preferred. Current covalent protein attachment methods use complex wet chemical approaches such as multi-step linker chemistry processes which are limited to compatible proteins and surface chemistries and often result in incomplete or side reactions leaving potentially toxic residues.

Commercial lab suppliers offer ready-to-use glass coverslips coated with poly-D-lysine, collagen, laminin, gelatine or fibronectin. Although they afford time saving options, these products are expensive and provide little flexibility to tune the surface required for different cell culture experiments or to coat glass surfaces with alternative proteins.

The development of stem cell research in recent decades has revolutionised regenerative medicine and personalized cell therapy, bringing hope for patients who do not respond to conventional therapies or with rare diseases (N. Rajabzadeh, E. Fathi, R. Farahzadi, Stem Cell Investig 2019, 6, 19). In this regard, pluripotent embryonic stem (ES) cells and patient-derived induced pluripotent stem (iPS) cells are useful tools for developing high-throughput drug screens and novel regenerative medical interventions. While iPS cell lines are a fairly recent development, mouse ES cells have been available since the early 1980s to study pluripotency and in vitro differentiation. Mouse ES cells can be maintained in vitro in the stem cell-state by the addition of exogenous leukemia inhibitory factor (LIF) and upon withdrawal of LIF spontaneously differentiate into cell lineages of all three germ layers: the ectoderm, the mesoderm and the endoderm (G. Keller, Genes & development 2005, 19, 1129). The ectoderm gives rise to skin and central nervous system neural lineages; the mesoderm gives rise to bone, blood and muscle while the endoderm gives rise to epithelial organs of the respiratory, digestive and genitourinary tracts. The differentiation pathways of mouse ES cells into a multitude of cell types have been well characterised, See, for example, a) A. Waisman, F. Sevlever, M. Elías Costa, M. S. Cosentino, S. G. Miriuka, A. C. Ventura, A. S. Guberman, Scientific Reports 2019, 9, 8051; b) B. B. Boumelhem, S. T. Fraser, S. J. Assinder, Methods in Molecular Biology (Clifton, N.J.) 2019, 2029, 103; c) H. Wichterle, I. Lieberam, J. A. Porter, T. M. Jessell, Cell 2002, 110, 385; d) Q.-L. Ying, M. Stavridis, D. Griffiths, M. Li, A. Smith, Nature Biotechnology 2003, 21, 183; e) M. Bibel, J. Richter, K. Schrenk, K. L. Tucker, V. Staiger, M. Korte, M. Goetz, Y.-A. Barde, Nature Neuroscience 2004, 7, 1003.

Cultivation of pluripotent stem cells in vitro requires mimicking the factors which closely resemble the embryonic and fetal microenvironments of the target cell lineage. This includes cell signalling events, biochemical elements and physical cues (P. Zhang, C. Zhang, J. Li, J. Han, X. Liu, H. Yang, Stem Cell Research & Therapy 2019, 10, 327). The cell culture substrate plays a critical role in cell-cell signalling, regulation and maintenance of growth as well as the differentiation of cells attached (A. A. Khalili, M. R. Ahmad, Int J Mol Sci 2015, 16, 18149). Apart from the surface immobilized extracellular matrix (ECM) required for conventional cell culture, some niche factors are important to drive the cell fate (F. Gattazzo, A. Urciuolo, P. Bonaldo, Biochim Biophys Acta 2014, 1840, 2506). Substrate tensile strength can also impact the differentiation outcome (A. J. Engler, S. Sen, H. L. Sweeney, D. E. Discher, Cell 2006, 126, 677). Covalent attachment of those molecules on cell culture substrates is preferred to reduce their loss during media changing and other washing steps. Transparency of the substrate may also be very important for subsequent spectroscopic imaging purposes. Therefore, ideal substrates for stem cell differentiation should provide two characteristics: they should be optically clear and also strongly retain ECM and niche factors.

Accordingly, a need exists to provide modified substrates and processes for their preparation that address one or more of the aforementioned shortcomings.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE DISCLOSURE

Disclosed herein are new coated substrates and processes for their preparation. The coated substrates comprise a plasma-activated coating containing radical concentrations which enable covalent attachment of biomolecules or other molecules to the coated substrate.

In one aspect the present disclosure provides a coated glass, quartz or silicon substrate, wherein the coating comprises a plasma-activated coating, said plasma-activated coating comprising radicals, and wherein the coated substrate is characterised by one or more of the following features:
a) a plasma-activated coating thickness from about 1 nm to about 100 nm;
b) a UV/visible absorbance in a range from about 320 nm to about 600 nm which is substantially the same as that of the substrate absent the plasma-activated coating;
c) an electron spin resonance peak centred at an applied magnetic field strength in a range from about 333 mT to about 339 mT;
d) electron spin resonance peaks centred at an applied magnetic field strength in a range from about 327 mT to about 332 mT and in a range from about 340 mT to about 345 mT;
e) a water contact angle of less than 60° measured within 1 day of preparation of the coated substrate, wherein the coated substrate is stored at ambient conditions prior to measurement;
f) a water contact angle of less than 80° measured within three months of preparation of the coated substrate, wherein the coated substrate is stored at ambient conditions prior to measurement;
g) a nitrogen:carbon elemental ratio in the coating of about 0.01:1 to about 1:1;
h) infrared absorption peaks in the range 1400-1800 cm⁻¹; and
i) an inability to remove all plasma-activated coating from the substrate with rigorous washing.

As used herein, ambient conditions refers to normal laboratory conditions in an atmosphere of air.

In embodiments, the plasma-activated coating thickness is from about 1 nm to about 100 nm, or from about 2 nm to about 50 nm, or from about 5 nm to about 40 nm.

In embodiments, the water contact angle after storage for 1 day under ambient conditions is less than 50°.

In embodiments, the water contact angle after storage for three months under ambient conditions is less than 70°.

In embodiments, the radical content of the plasma-activated coating decreases by less than 80% after storage for three months under ambient conditions.

In embodiments, the plasma-activated coating may have a high radical density.

In embodiments, the nitrogen:carbon elemental ratio in the plasma-activated coating is about 0.1:1 to about 2:3.

In embodiments, the atomic percentage of elemental nitrogen as measured by XPS in the plasma-activated coating is from about 50% to about 1%, or from about 40% to about 1%.

In embodiments, the atomic percentage of elemental carbon as measured by XPS in the plasma-activated coating is from about 90% to about 10%, or from about 70% to about 10%.

In embodiments, the washing in feature (i) is performed with an aqueous detergent solution, preferably at elevated temperature. For example, washing with 5% sodium dodecyl sulphate (SDS) at, for example, 70° C.

In embodiments, the coated substrate is substantially transparent.

Generally, the type of glass is not critical. In embodiments the glass substrate may comprise silicate glass, borosilicate glass, soda-lime glass or silicon oxide. In some preferred embodiments the glass substrate comprises borosilicate glass.

The glass substrate may be of any shape or form. In some embodiments, the glass substrate is a glass coverslip, a flat-bottomed glass dish or glass cell culture chamber slide.

In embodiments, the herein disclosed plasma-activated coated substrates, further comprise one or more hydrogels covalently bonded to the plasma activated coating.

In embodiments, the hydrogels comprise at least two layers, wherein a first layer of hydrogel is derived from reacting hydrogel monomers with the plasma-activated coating and the second layer of hydrogel is derived from polymerising hydrogel monomers, said second layer of hydrogel being disposed atop the first layer of hydrogel. In embodiments, the second layer of hydrogel is covalently bonded to or immobilised on the first layer of hydrogel.

Suitable hydrogels include those which are crosslinkable through radical initiation.

Suitable hydrogels include peptide and protein based hydrogels.

Examples of suitable hydrogels include those derived from acrylated polyvinyl alcohol, such as, for example, methacrylated polyvinyl alcohol. Other suitable hydrogels include GelMA (gelatin+methacrylic anhydride), polyacrylamide, silk hydrogel, hyaluronic acid hydrogels, and beta-peptide hydrogels modified with cell adhesive sequences such as RGD (arginylglycylaspartic acid).

In another aspect, the present disclosure provides a biomolecule functionalised coated substrate comprising the coated substrate according to any one of the herein disclosed embodiments and one or more biomolecules covalently bonded thereto.

In some embodiments, the biomolecules may be covalently bonded to the plasma activated coating.

In other embodiments, when hydrogel is present, the biomolecules may be covalently bonded to the hydrogel.

In embodiments, the one or more biomolecules may comprise one or more proteins, polysaccharides, nucleotides, oligonucleotides, antioxidants, growth factors, vitamins or lipids. In embodiments, the one or more biomolecules comprise glycoproteins.

In embodiments, the one or biomolecules comprise laminin. In embodiments, the laminin comprises laminin-521.

In embodiments, the biomolecule functionalised coated substrate comprises extracellular matrix covalently bonded to the coating or immobilised on the coating.

In embodiments, the biomolecule functionalised coated substrate comprises niche factors covalently bonded to the coating or immobilised on the coating.

In embodiments, the biomolecule functionalised coated substrate comprises cells covalently bonded to the coating or immobilised on the coating.

In embodiments, the biomolecule functionalised coated substrates are characterised by an inability to remove all biomolecules from the coated substrate with rigorous washing.

In embodiments, the washing is performed with an aqueous detergent solution, preferably at elevated temperature. For example, washing with 5% sodium dodecyl sulphate (SDS) at, for example, 70° C.

For the avoidance of doubt, reference to coated substrate may include both plasma-activated coatings with and without further hydrogel coating.

In another aspect the present disclosure provides a process for producing a plasma-activated coated substrate, comprising:
a) activating one or more surfaces of a substrate by exposing said substrate to a plasma formed in the presence of one or more of helium, neon, argon and xenon; and
b) depositing a plasma-activated coating on the one or more activated surfaces of the substrate by exposing said activated surface to a plasma formed in the presence of one or more organic gases, and one or more of nitrogen, helium, neon, argon and xenon;
   wherein the total pressure in step b) is from about 25 mTorr (3.3 Pa) to about 500 mTorr (66.7 Pa).

In embodiments, the total pressure in step a) is from about 25 mTorr (3.3 Pa) to about 500 mTorr (66.7 Pa).

In embodiments, the total pressure in step b) is from about 25 mTorr (3.3 Pa) to about 350 mTorr (46.7 Pa), or from about 25 mTorr (3.3 Pa) to about 250 mTorr (33.3 Pa), or from about 25 mTorr (3.3 Pa) to about 150 mTorr (20.1 Pa),

In embodiments, the total pressure in step b) is less than about 250 mTorr (33.3 Pa), or less than about 200 mTorr (26.7 Pa).

In embodiments, a plasma discharge in step a) is maintained from about 10 seconds to about 30 minutes, or from about 2 minutes to about 20 minutes.

In some embodiments, a plasma discharge in step a) is maintained for about 5 minutes. In other embodiments, a plasma discharge in step a) is maintained for about 20 minutes.

In embodiments, a plasma discharge in step b) is maintained from about 30 seconds to about 30 minutes, or from about 2 minutes to about 20 minutes.

In embodiments, the plasma in step a) is formed in the presence of argon.

In embodiments, the plasma in step b) is formed in the presence of nitrogen and one or more of helium, neon, argon and xenon.

In embodiments, the plasma in step b) is formed in the presence of nitrogen and argon.

In embodiments, the one or more organic gases comprise one or more of hydrocarbon and substituted hydrocarbon.

In embodiments, the one or more organic gases comprises one or more linear or branched alkane or cycloalkane, linear or branched alkene or cycloalkene, and linear or branched alkyne.

In embodiments, the one or more organic gases comprises acetylene or substituted acetylene.

In embodiments, the substituted hydrocarbon comprises one or more of hydroxyl substituted hydrocarbon, amino substituted hydrocarbon, and hydrocarbons substituted with sulphur-containing groups.

In embodiments, the power supplied in step a) and step b) is in the form of DC power, pulsed DC power, or AC power, such as RF power or microwave power.

In embodiments, the power supplied in step a) is from about 10 W to about 200 W, or from about 50 W to about 100 W.

In embodiments, the power supplied in step b) is from about 10 W to about 200 W, or from about 25 W to about 100 W.

In embodiments, during step (a), a pulsed bias of from about −200 V to about −1000 V, with a frequency from about 500 Hz to about 5000 Hz, and a pulse length from about 5 μs to about 20 μs, may be applied. Other combinations of bias voltage, frequency and pulse length are contemplated.

In embodiments, during step (b), a pulsed bias of from about −200 V to about −1000 V, with a frequency from about 500 Hz to about 5000 Hz, and a pulse length from about 5 μs to about 20 μs, may be applied. Other combinations of bias voltage, frequency and pulse length are contemplated.

In embodiments, the volume ratio of the at least one organic gas fed to the plasma chamber to the sum of one or more of nitrogen, helium, neon, argon and xenon, is about 1 to 20 to about 1 to 2, or from about 1 to 15 to about 1 to 5.

In embodiments, the volume ratio of the at least one organic gas fed to the plasma chamber to the sum of nitrogen and argon is about 1 to 20 to about 1 to 2, or from about 1 to 15 to about 1 to 5.

The inventors have discovered that the electron spin resonance peaks centred at an applied magnetic field strength in a range from about 327 mT to about 332 mT and in a range from about 340 mT to about 345 mT are both present after step (a) of the herein disclosed process.

It is envisaged that these electron spin resonance peaks are due to substrate radicals resulting from ion bombardment and are important in providing good coating adhesion onto the substrate when step (b) is performed.

Further, biomolecule attachment to the coated substrates may occur when an unpaired electron of an embedded radical migrates to the surface and because radicals may be lost through reactions when they reach the surface, deep radicals last longer and are therefore advantageous. This means that a thicker coating containing radicals throughout its depth may achieve longer active shelf life for the coated substrates.

Hydrogels are useful materials for modulating the stiffness of the surface as sensed by cells and for the covalent attachment of biomolecules or other species.

Accordingly, in another aspect the present disclosure provides a process for producing a coated substrate comprising hydrogels, comprising the step of contacting one or more hydrogel monomers with the plasma-activated coated substrate according to any one of the herein disclosed embodiments.

Without wishing to be bound by theory, it is envisaged that radicals within the plasma-activated coating facilitate polymerisation of hydrogel monomers to provide hydrogel covalently bonded to the plasma-activated coating.

In embodiments, the process further comprises the step of contacting one or more hydrogel monomers and initiator with the coated substrate comprising hydrogels.

Without wishing to be bound by theory, it is envisaged that the radical content in the plasma-activated coating may, in embodiments, be low, particularly for very thin plasma-activated coating thickness and this may not be sufficient to maintain polymerisation for long enough to form a thick hydrogel. However, this very thin hydrogel layer grows from the plasma-activated coating (with multiple covalent bonds) and therefore strongly attaches to the substrate underneath. The thicker hydrogel layer formed through initiator (for example, photoinitiator) then forms on top of the initial hydrogel layer (interfacial layer) and covalently binds to the interfacial layer (due to the radical supply from photoinitiator) and thus, the whole structure builds on a strong foundation which may survive shear forces, such as from washing.

In another aspect the present disclosure provides a process for covalently attaching biomolecules to the herein disclosed coated substrate, comprising the step of contacting one or more biomolecules with the coated substrate.

In embodiments, attachment may be achieved in 1 hour or less.

In embodiments, biomolecules may be attached across an entire surface or, alternatively, in patterns.

The one or more biomolecules may comprise one or more of proteins, polysaccharides, nucleotides, oligonucleotides, antioxidants, growth factors, vitamins or lipids.

In embodiments, the one or biomolecules comprise laminin. In embodiments, the laminin comprises laminin-521.

In another aspect the present disclosure provides a process for covalently attaching extracellular matrix to the herein disclosed coated substrate, comprising the step of contacting extracellular matrix with the coated substrate.

In another aspect the present disclosure provides a process for covalently attaching niche factors to the herein disclosed coated substrate, comprising the step of contacting niche factors with the coated substrate.

In another aspect the present disclosure provides a process for covalently attaching cells to the herein disclosed coated substrate, comprising the step of contacting cells with the coated substrate.

In another aspect the present disclosure provides a composition of matter comprising a biomolecule functionalised coated substrate according to any one of the herein disclosed embodiments and one or more cells.

In another aspect, the present disclosure provides a method of cell differentiation comprising:
(a) depositing pluripotent cells in a cell culture medium on a plasma-activated coated substrate, or a plasma-activated coated substrate further comprising hydrogel, according to any one of the herein disclosed embodiments;
(b) covalently attaching biomolecules from the cell culture medium to the plasma-activated coating or hydrogel; and
(c) allowing the cells to differentiate;
wherein the differentiation is directed by the covalently attached biomolecules and/or hydrogel.

In embodiments, the one or biomolecules comprise laminin. In embodiments, the laminin comprises laminin-521.

In another aspect, the present disclosure provides a method of cell differentiation comprising:
(a) depositing biomolecules on a plasma-activated coated substrate, or a plasma-activated coated substrate further comprising hydrogel, according to any one of the herein disclosed embodiments;
(b) covalently attaching one or more of the biomolecules to the plasma-activated coating and/or hydrogel;
(c) depositing pluripotent cells in a cell culture medium on the plasma-activated coating and/or hydrogel; and
(d) allowing the cells to differentiate;
wherein the differentiation is directed by the covalently attached biomolecules and/or hydrogel.

In embodiments, the one or more biomolecules comprise laminin. In embodiments, the laminin comprises laminin-521.

In another aspect, the present disclosure provides a method of attaching cells to a substrate comprising:
(a) depositing cells in a cell culture medium on a plasma-activated coated substrate, or a plasma-activated coated substrate further comprising hydrogel, according to any one of the herein disclosed embodiments;
(b) covalently attaching biomolecules from the cell culture medium to the plasma-activated coating and/or hydrogel; and
(c) attaching the cells to the covalently attached biomolecules and/or hydrogel.

In another aspect, the present disclosure provides a method of attaching cells to a substrate comprising:
(a) depositing biomolecules on a plasma-activated coated substrate, or a plasma-activated coated substrate further comprising hydrogel, according to any one of the herein disclosed embodiments;
(b) covalently attaching one or more of the biomolecules to the plasma-activated coating and/or hydrogel;
(c) depositing cells in a cell culture medium on the plasma-activated coating and/or hydrogel; and
(d) attaching the cells to the covalently attached biomolecules and/or hydrogel.

In another aspect, the present disclosure provides a method of increasing cell survival on a substrate comprising:
(a) depositing cells in a cell culture medium on a plasma-activated coated substrate, or a plasma-activated coated substrate further comprising hydrogel, according to any one of the herein disclosed embodiments;
(b) covalently attaching biomolecules from the cell culture medium to the plasma-activated coating and/or hydrogel; and
(c) attaching the cells to the covalently attached biomolecules and/or hydrogel.

In another aspect, the present disclosure provides a method of increasing cell survival on a substrate comprising:
(a) depositing biomolecules on a plasma-activated coated substrate, or a plasma-activated coated substrate further comprising hydrogel, according to any one of the herein disclosed embodiments;

(b) covalently attaching one or more of the biomolecules to the plasma-activated coating and/or hydrogel;
(c) depositing cells in a cell culture medium on the plasma-activated coating and/or hydrogel; and
(d) attaching the cells to the covalently attached biomolecules and/or hydrogel.

Any one or more of the hereinbefore disclosed methods may include one or more of improved cell differentiation, improved cell attachment, improved cell survival, improved cell proliferation and improved cell function.

In any one or more of the hereinbefore disclosed methods, the cells may comprise pluripotent cells.

In any one or more of the herein disclosed embodiments, the one or biomolecules comprise laminin. In embodiments, the laminin comprises laminin-521.

In any one or more of the herein disclosed embodiments the substrate may be a glass substrate, a quartz substrate or a silicon substrate, such as a silicon wafer.

In any one or more of the herein disclosed embodiments, the coated substrate may include hydrogels.

Advantages of the presently disclosed coated substrates may include one or more of the following: plasma-activated coating (PAC) provides a rapid and simple two-step process for covalent attachment of any biomolecule to a modified surface without using chemical linkers. In embodiments, the coating may be thin, strongly adherent to the substrate, and radical-rich, enabling the direct attachment of biomolecules from solutions by reactions with embedded radicals. Biomolecule attachment using PAC requires no chemical modifications to either the modified surface or biomolecule. PAC coatings may be engineered for improved biomolecule attachment by altering the chemical composition of the PAC layer in the plasma process via regulating the input gas mixture. This may alter the surface charge of the coating to favour the attachment of different biomolecules. PAC surfaces may be decorated with defined biomolecule combinations to promote lineage specific differentiation of stem cells or induce specific cell phenotypes. In embodiments, PAC has minimal impact on visible light transmission making it ideal for live cell imaging or any other optical observation technique. Overall, the herein disclosed PAC technology offers the possibility to individually tailor cell culture surfaces on previously inhospitable surfaces at reduced cost and with far greater flexibility of biomolecule choice than previously offered, without the need for complex, often toxic, linker based wet chemistry.

Further aspects of the present disclosure and further embodiments of the aspects described in the preceding paragraphs will become apparent from the following description, given by way of example and with reference to the accompanying drawings.

### BRIEF DESCRIPTION OF THE DRAWINGS

FIG. 1 compares optical absorbances of PACs in the UV and visible wavelength range with a cleaned glass cover slip (denoted as glass) and polystyrene (denoted as PS).

FIG. 2 compares water contact angles and surface energies, including their dispersive and polar components calculated using the Owens-Wendt-Rabel-Kaeble method. Glass and polystyrene (PS) are used as controls.

FIG. 3 is XPS analysis of PACs on silicon wafer. (A) Atomic percentages of elements detected on the surfaces. (B) Percentage of deconvoluted peaks from C1s high resolution peaks. Deconvolution of C1s obtained from No_N (C) and High_N (D) coatings showing the status of carbon bonds.

FIG. 4 is FTIR spectra of PACs, identifying some functional groups from the absorbances in the mid-infrared range.

FIG. 5 compares electron spin resonance measurements. (A) First derivative of EPR signal of Low_N coating just after the treatment, 1 week and 3 months, showing radical decay of peak 2 over time. (B) Comparison of peak 2 area during storage time measured on four PACs.

FIG. 6 compares microcontact printing of fluorescently labelled gelatine on bare glass (UT) and PAC using confocal microscopy. (A) Comparative fluorescent micrographs at low resolution (upper micrographs) and high resolution (lower micrographs). Images are based on fluorescence intensity; the far right hand image (UT SDS wash) has very low fluorescence intensity; the UT H2O wash has poor edge fidelity; the PAC samples have improved edge fidelity; (B) Mean fluorescent intensity from high resolution images, x-axis indicates distance from the left edge of each image.

FIG. 7 compares ES differentiation on glass cover slips (Control) and PACs. (a) Total number of clusters. (b) Total number of neurites. (c) Average size of neurites. Groups not sharing a number are significantly different from each other.

FIG. 8 illustrates Mouse ES cells attached to PAC glass coverslips preferentially differentiate into neuronal-like cells. Scale bars indicate a length of 200 μm. ** denotes surface ectoderm differentiation while white arrowheads denote neural cell differentiation.

FIG. 9 shows Venn diagrams comparing the number of shared versus unique proteins on untreated glass and all PAC recipes before (A) and after (B) washing with 5% sodium dodecyl sulphate (SDS). All surfaces showed a decrease in the total number of proteins present after washing. On post washed samples the increasing concentrations of nitrogen in the PAC correlate with a decrease in the total amount of protein detected along with fewer types of protein.

FIG. 10 contains scatter plots comparing the molecular weight (kDa), charge (mV) of the highest ranked proteins for each recipe. The size of each marker is relative to the total peptide spectral matches for each protein. Albumin and serotransferrin PSM values are the sum of both human and bovine sources due to the high degree of similarity of the proteins.

FIG. 11 shows sample layer compositions for photoinitiator experiments. (a) and (c) show samples prepared with 10 w/w % PVA-MA with photoinitiator solution directly onto bare silicon wafer (a) or PAC treated silicon wafer (c). (b) and (d) show samples prepared with 10% w/w PVA-MA with photoinitiator solution onto intermediate layer of either 1% PVAMA or MQ water incubated under continuous flow of argon. Relative sizes of layers are not to scale.

FIG. 12 shows Electron Paramagnetic Resonance (EPR) intensities measured on PAC treated quartz slides before and after incubation with milli-Q water (two largest bars) or 1 w/w % PVA solution (two smallest bars). Error bars indicate 8% error from the mean value (n=5).

FIG. 13 contains photographs before and after reswelling experiments for 10% PVA-MA with photoinitiator solution incubated directly on PAC or untreated samples (which lacked intermediate PVA-MA layer incubated under argon continuous flow).

FIG. 14 compares plasma coating components from XPS analysis of samples before and after soaking in simulated body fluid (SBF) for 4 weeks. Data are the average of 3 random spots on the coatings.

FIG. 15 shows surface analysis of silicon wafer samples after treatment with Mid Nitrogen plasma; (a) and (b) XPS data showing the relative percentages of carbon, nitrogen, oxygen and silicon detected on the surface of the samples (a) before SDS and (b) after SDS washing; (c) ellipsometry data showing the relative thicknesses of the plasma layer on the silicon wafer samples after various times of argon etching.

FIG. 16 is an XPS surface analysis of silicon wafers with immobilised Laminin-521. Percentage of sulphur detected is relative to the silicon wafer surface as a whole and is representative of the Laminin-521 detected.

FIG. 17 shows brightfield images of iPSC-CM differentiation over 17 days; (a) Day 2, end of differentiation media A, start of media B; (b) Day 4, end of differentiation media B, start of media C; (c) Day 10, 2 days into the maintenance media; and (d) Day 17, 10 days into the maintenance media.

FIG. 18 shows (a) relative percentage of beating cardiomyoctes observed over the culture period as a proportion of the whole coverslip for PAC treated and untreated coverslips with Laminin-521; (b) relative cardiomyocyte beat rate (in beats per minute) over the culture period for both PAC-treated samples.

### DETAILED DESCRIPTION OF THE EMBODIMENTS

Reference will now be made in detail to certain embodiments of the present disclosure. While the disclosure will be described in conjunction with the embodiments, it will be understood that the intention is not to limit the disclosure to those embodiments. On the contrary, the disclosure is intended to cover all alternatives, modifications, and equivalents, which may be included within the scope of the present disclosure as defined by the claims.

One skilled in the art will recognize many methods and materials similar or equivalent to those described herein, which could be used in the practice of the present disclosure. The present disclosure is in no way limited to the methods and materials described. It will be understood that the disclosure described and defined in this specification extends to all alternative combinations of two or more of the individual features mentioned or evident from the text or drawings. All of these different combinations constitute various alternative aspects of the disclosure.

All of the patents and publications referred to herein are incorporated by reference in their entirety.

For the purposes of interpreting this specification, terms used in the singular will also include the plural and vice versa.

Unless defined otherwise, all technical and scientific terms used herein have the same meaning as commonly understood by those of ordinary skill in the art to which the present disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e. to at least one) of the grammatical object of the article. By way of example, “an organic gas” means one organic gas or more than one organic gas.

As used herein, the term “and/or”, e.g., “X and/or Y” will be understood to mean either “X and Y” or “X or Y” and shall be taken to provide explicit support for both meanings or for either meaning.

As used herein, the term “about” refers to a quantity, value, dimension, size, or amount that varies by as much as 10%, 5%, 1% or 0.1% to a reference quantity, value, dimension, size, or amount.

Throughout the present disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, unless the context requires otherwise, the term “comprise”, and variations such as “comprises” and “comprising”, will be understood to imply the inclusion of a stated integer or step or group of integers or steps but not the exclusion of any other integer or step or group of integers or steps.

It will be understood that the disclosure described and defined in this specification extends to all alternative combinations of two or more of the individual features mentioned or evident from the text. All of these different combinations constitute various alternative aspects of the disclosure.

The present disclosure relates to new coated substrates and processes for their preparation. The coated substrates comprise a plasma-activated coating which contains radical concentrations enabling covalent attachment of biomolecules to the coated substrate.

### Process for Preparing Coated Substrates

In embodiments, substrates were coated with plasma-activated coating in a two-step process. The process may be conveniently performed in a plasma chamber or plasma reactor. The substrate was placed on a stainless-steel sample holder inside the plasma chamber which was then evacuated to low pressure. In a first process step, plasma may be generated using a capacitively coupled radio frequency generator with a power of, for example, 75 W, for about 10 minutes in low pressure argon. During this surface activation step, a pulsed bias of −500V, with a frequency of 3000 Hz and 20 μs pulse length, was applied to the sample holder. The chamber was again evacuated to the base pressure and then a mixture of gases including, for example, acetylene, nitrogen and argon was introduced into the chamber. Pressure during deposition was kept constant at, for example 110 mTorr. The deposition occurred for about 10 minutes at 50 W radio frequency power while the same negative pulsed bias was applied. After plasma coating, samples were stored in a petri dish in ambient laboratory conditions for further analysis.

The capacitively coupled radio frequency power used to generate the plasma may have a power of about 5 to about 500 W, or about 5 to 100, 5 to 200, 5 to 300, or 5 to 400 W, e.g., about 5, 10, 20, 30, 40, 50, 60, 70, 80, 90, 100, 120, 140, 160, 180, 200, 250, 300, 350, 400, 450 or 500 W.

The pulsed bias voltage may have a frequency of about 1 Hz to about 50 kHz, or about 1 Hz to 20 KHz, 1 Hz to 10 KHz, 1 Hz to 5 kHz, 5 Hz to 50 kHz, 10 Hz to 50 kHz, 20 Hz to 50 kHz, 10 Hz to 20 kHz or 20 Hz to 30 KHz.

The bias voltage may be from about −1000V to about 1000 V, or −500 to 500, −200 to 200, −100 to 100, −50 to 50, −1000 to 0, −500 to 0, −200 to 0, −100 to 0, −50 to 0, 0 to 50, 0 to 100, 0 to 200, 0 to 500 or 0 to 1000V, e.g., about −1000, −900, −800, −700, −600, −500, −400, −300, −200, −100, −50, 0, 50, 100, 200, 300, 400, 500, 600, 700, 800, 900 or 1000 V. In embodiments, negative bias voltages are preferred, for example, from about −100 V to about −1000 V, or from about −200 V to about −1000 V.

The pulse duration may be from about 1 to about 150 microseconds, or about 1 to 100, 1 to 50, 1 to 20, 1 to 10, 10 to 150, 20 to 150, 50 to 150, 100 to 150, 10 to 100, 10 to 50 or 50 to 100 microseconds, e.g., about 1, 2, 3, 4, 5, 10, 15, 20, 25, 30, 35, 40, 45, 50, 60, 70, 80, 90, 100, 110, 120, 130, 140 or 150 microseconds.

The ratio between off time and on time of the pulses may be from about 1 (i.e., 1:1) to about 20, or about 10 to 15, 15 to 20 or 13 to 17, e.g., about 10, 13, 15, 17 or 20. In some instances it may be greater than 10, optionally up to 100, or up to 90, 80, 70, 60, 50, 40 or 30.

The gas comprises at least one gas which is organic, i.e., contains carbon and is not carbon dioxide. It may comprise a carbon-carbon double bond and/or a carbon-carbon triple bond. It may be an alkene or an alkyne. It may be a mixture of such gases. The organic gas may be polymerisable under the conditions of the process. The gas may comprise more than one organic gas. In the present context, a gas is taken to be a substance which is in the gaseous state at the temperature and pressure prevailing at the time in the plasma chamber.

The flow rates of gases into the plasma chamber may be varied to achieve a desired pressure and residence time within the chamber. Flow rates are dependent on the scale of operation and the size of the plasma chamber.

The deposition time may be from less than 1 minute to about 30 minutes, or from 1 to 20, 1 to 10, 1 to 50, 5 to 30, 10 to 30, 20 to 30, 5 to 20, 5 to 10 or 10 to 20 minutes, e.g., about 1, 2, 3, 4, 5, 6, 7, 8, 9, 10, 15, 20, 25 or 30 minutes or may be longer than 30 minutes.

It will be appreciated that any of the flow rate, pressure and power may be varied according the specifically desired properties of the coated substrate to be produced. Thus, any of the numerical values or ranges exemplified herein for each of the pressure and power may be used together, in any combination. For example, in one embodiment of the deposition step, a pressure of about 20 Pa and a power of about 50 W to about 100 W may be used. All other possible combinations are envisaged herein.

### Coated Substrates

One key advantage of the coated substrates described herein is that they may retain radicals embedded in them for an extended period of time. When the materials are stored in appropriate conditions the radicals may be retained for weeks, months or potentially years. The extended lifetime of the radicals means that the coated substrates may be stored until such time that they are required to be used. This means that production of the coated substrate need not occur at the same time as biomolecule attachment is performed.

Coated substrates may be formed in the presence of nitrogen and fragments of this gas may be imported into the coating. For example, the use of nitrogen may result in the presence of amine, imine or nitrile groups, or a mixture thereof in the coating. Thus, the coating disclosed herein may comprise nitrogen.

The coating may have a nitrogen:carbon elemental ratio of about 0.01:1 to about 1:1. For example the coating may have a nitrogen:carbon elemental ratio of about 0.1:1 to about 1:1, or about 0.15:1 to about 1:1, or about 0.2:1 to about 1:1, or about 0.25:1 to about 1:1, or about 0.3:1 to about 1:1, or about 0.35:1 to about 1:1, or about 0.4:1 to about 1:1, or about 0.45:1 to about 1:1, or about 0.5:1 to about 1:1, or about 0.55:1 to about 1:1, or about 0.6:1 to about 1:1, or about 0.65:1 to about 1:1.

The coating may comprise unpaired electrons. These unpaired electrons may be on or near the surface of the coating. The unpaired electrons may be at a depth of 40 nm or less from the surface of the coating, or within about 30, 20 or 10 nm of the surface, or may be from about 10 to about 40 nm from the surface, or from about 10 and 30, 20 and 40, or 20 and 30 nm from the surface, or about 1, 2, 3, 4, 5, 10, 15, 20, 25, 30, 35 or 40 nm from the surface. They may be at a variety of depths from about 0 to about 40 nm. In some instances they may be at depths of greater than 40 nm. They may be throughout the volume of the coating. This may render the coating capable of reacting with another material, such as a biomaterial, so as to covalently bond said material to the coating.

Four coated glass substrates were prepared using varying ratios of acetylene, nitrogen and argon as summarised in Table 1.

TABLE 1

| Recipe | Acetylene (sccm) | Nitrogen (sccm) | Argon (sccm) |
|---|---|---|---|
| No_N | 1 | 0 | 13 |
| Low_N | 1 | 3 | 10 |
| Mid_N | 1 | 10 | 3 |
| High_N | 1 | 13 | 0 |

sccm = standard cubic centimetres per minute

### Optical and Chemical Properties of PAC Coated Glass Coverslips

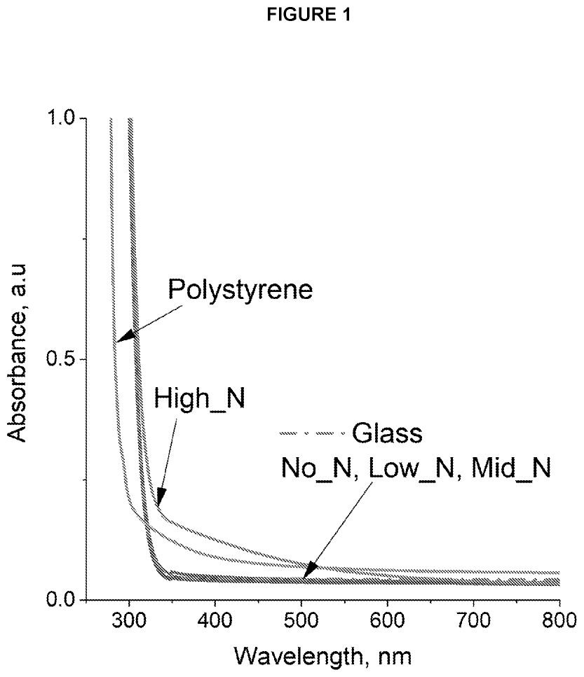

Transparency of glass coverslips plays an important role in the microscopy image quality. It was found that employing the same deposition process parameters but with varied gas mixture composition, PACs had different thicknesses but were very thin compared to the glass coverslip thickness. Thickness of the No_N and Low_N coatings varied between 10-15 nm, Mid_N coating varied between 15-20 nm while the High_N coating had thickness varying between 20-30 nm. The absorbances of the PACs on glass cover slips in the UV and visible wavelength range were compared with those of a cleaned glass cover slip and polystyrene film (0.19 mm) (FIG. 1). All PACs, that is No_N, Low_N, and Mid_N, had the same absorbance values as the cleaned glass cover slip and lower than polystyrene, except for High_N which had a higher absorbance in 320-600 nm range. These visible and near UV ranges are important for fluorescence staining such as DAPI (excitation at 351 nm, emission at 461 nm) and fluorescein-5-isothiocyanate (excitation at 491 nm, emission at 516 nm).

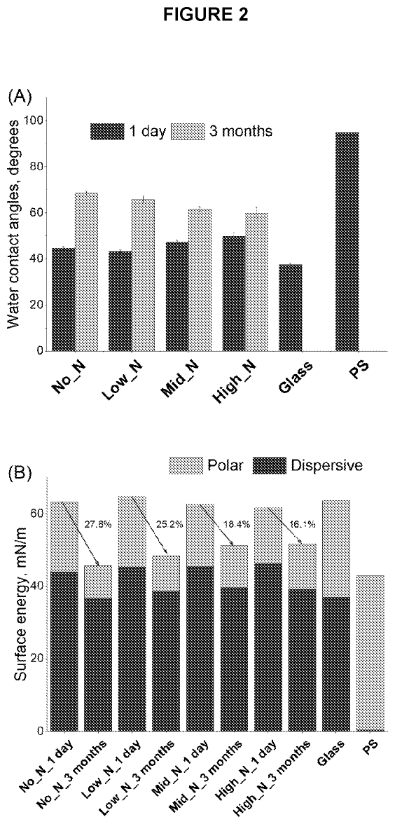

Surface wettability has a great impact on conformation of immobilized proteins. All PAC coated glass coverslips had water contact angles ranging between 40-50° (FIG. 2A) which is slightly higher than cleaned glass, but very low compared to polystyrene (94°). Among them, the High_N recipe had the highest contact angle. Their total surface energies (FIG. 2(B)) were close to clean glass surface but the polar energy was lower while the dispersive component of the surface energy was higher than that of glass. After 3 months stored under ambient conditions, the water contact angles on all PAC coatings increased to 60-68° while the total surface energy reduced by between 16.1% and 27.6%. The trend of surface energy reduction appeared to correlate with nitrogen levels in the plasma.

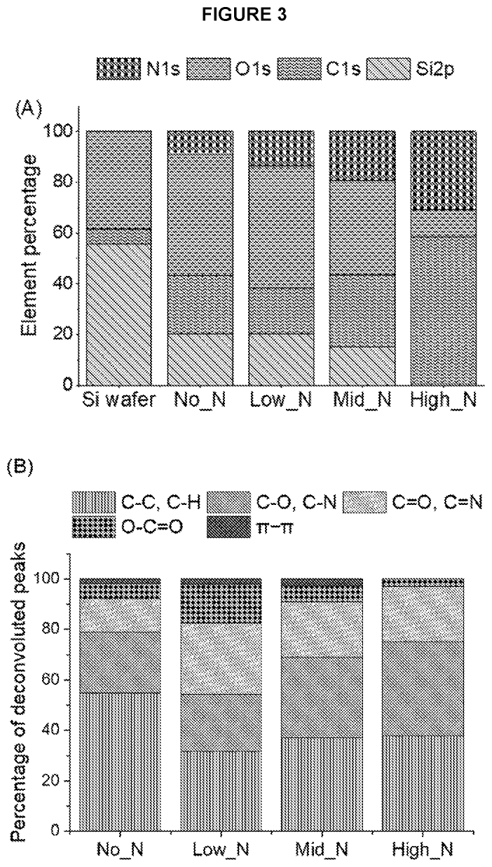

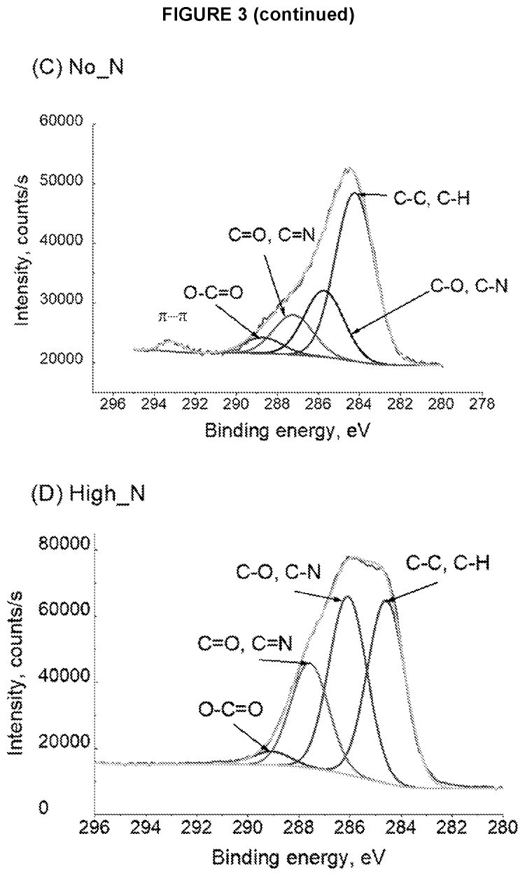

XPS analysis was conducted to compare the elemental composition of the coatings deposited on silicon wafers (FIG. 3). On a bare silicon wafer, apart from silicon and oxygen, a small percentage of carbon was detected, likely from a thin layer of adventitious carbon from environmental contamination. Silicon wafers always have a native oxide layer (approximately 2 nm thick) where oxygen was detected. XPS analysis on the PAC coated silicon wafers showed a small signal of silicon on all samples except for the High_N sample. This is likely because the High_N coating is thicker than the other coatings and XPS is most sensitive to approximately the top 10 nm of the sample. For those samples where silicon was detected, the oxygen may have originated from the PAC layer and the SiO₂ layer underneath. Nitrogen is the only element that is contained in the plasma coating alone. FIG. 3A shows that nitrogen content increases from the left to the right, corresponding to the increasing levels of nitrogen used in the gas mixtures. The maximum nitrogen level is 30% obtained from deposition in a gas mixture of acetylene and nitrogen only. This coating also has the highest carbon content and lowest oxygen content (far right hand bar in FIG. 3(A)).

The low oxygen content is likely because the coating prepared from acetylene and nitrogen only (no argon) was the thickest coating and oxygen in the coating appears when radicals emerging from the coating react with oxygen in the atmosphere when they reach the surface. It will be appreciated that the thickness of all of the disclosed coatings may be adjusted by modifying the coating deposition time and therefore the oxygen content would also be modified. Hence the lowest oxygen and highest carbon are not features of the high nitrogen coating per se but rather features of the thickest coating. The high nitrogen coating is the thickest because the deposition time was held constant for all gas mixtures.

C1s peaks were further deconvoluted to obtain carbon bond types. FIGS. 3C and 3D show examples of C1s peaks from the No_N and High_N samples respectively. All C1s peaks contain 4 or 5 deconvoluted peaks correlated to carbon bonding to carbon, oxygen and nitrogen (FIG. 3B). Among those, Low_N has the highest percentage of O—C═O groups while High_N has the lowest. Surfaces with O—C═O groups tend to have a more negative charge in buffer solutions while those with more nitrogen tend to have a more positive charge in an appropriate buffer.

Accordingly, the surface chemistry of the coatings may be altered by changing gas composition during the plasma deposition, in particular the elemental percentage and functional groups. These factors greatly influence surface charge of the coatings and the subsequence interaction with biomolecules.

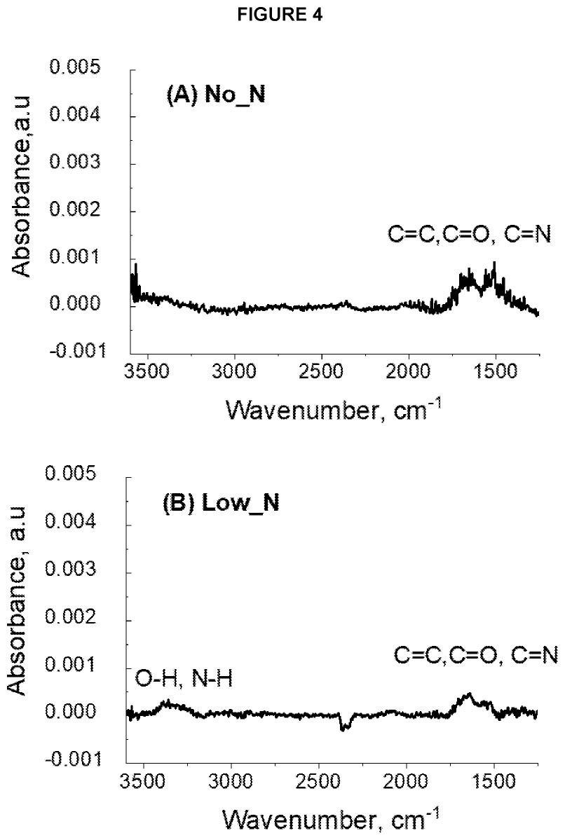

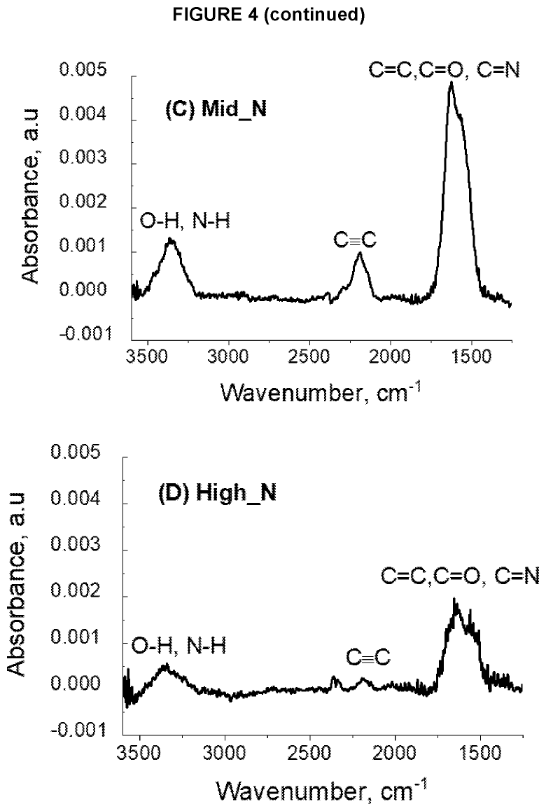

FTIR analysis (FIG. 4) further shows absorbances in 3600-3250 cm-1 correlating to vibrations of hydrogen bonds from O—H and N—H groups and in 1750-1500 cm-1 correlating to unsaturated carbonyl groups such as C═O, C═N and C═C. There is a small absorbance in 2200 cm-1 which is associated with C≡C bonds. This triple bond is not observed in No_N and Low_N coatings. This likely indicates a gas mixture with high argon can break down acetylene better than a low or no argon gas mixture.

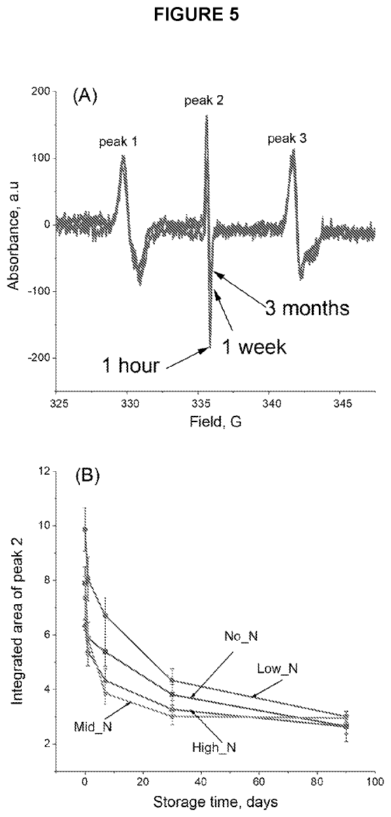

Radical density is an important factor influencing the covalent attachment of biomolecules on the PACs. EPR measurement detected three absorption peaks on quartz slides coated with PAC (FIG. 5A), among those, only peak 2 changed (reduced in intensity) over the storage time, while peaks 1 and 3 did not. Since the presently disclosed plasma treatment has two steps, surface activation using, for example, argon plasma and plasma deposition using a gas mixture, EPR was measured on quartz after the first step to identify the effect of argon plasma. It was found that peak 1 and peak 3 appeared with the same intensity as those detected on PAC coated quartz, while peak 2 was very small. Therefore, it is likely that peak 1 and 3 are radicals formed in the substrate (quartz) as the result of argon ion bombardment in the first process step while peak 2 mainly originated from the plasma deposition in the second process step.

FIG. 5B shows the reduction of peak 2 integrated area during storage under ambient conditions, correlating to the radical decay. The peak reduced sharply within the first day and continued decreasing in a month and then slows down after that. PAC with Low_N gas mixture had the highest radical content during the first month of storage while Mid_N and High_N samples had the lowest radical content. Despite the large difference in radical number just after the treatment, all PACs had relatively the same amount after 3 months. From this result, it can be seen that gas mixtures with high argon ratio (No_N and Low_N) produce thinner coatings but with higher number of radicals compared to those with high nitrogen ratios. This could be explained by the larger size and inertness of argon which has a bigger impact on ion bombardment (breaking bonds) without joining the surface structure (neutralize the radicals). A higher proportion of carbon also supports the retention of radicals as it enables the formation of a higher concentration of graphite-like (i.e. pi-coordinated) nanoclusters that have delocalised orbitals suitable for stabilising embedded radicals.

Nature of Protein Attachment on PAC in Comparison with Bare Glass

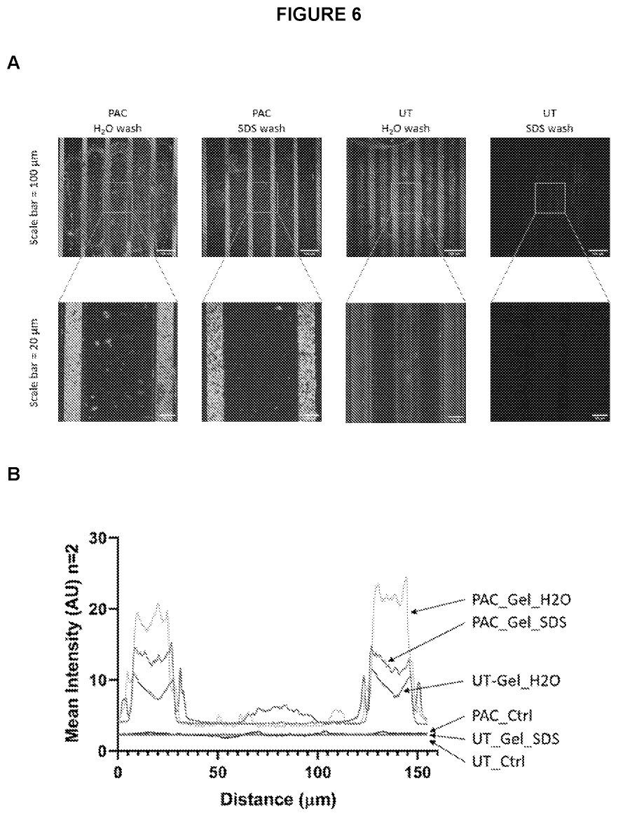

Microcontact printings of fluorescently labelled gelatine on the PAC and cleaned glass (denoted as UT) were compared after mild washing with water or stringent washing with SDS in FIG. 6. Gelatine on cleaned glass after washing with water showed poor edge fidelity as evidenced by the prominent shoulder regions flanking either side of the main peaks in FIG. 6B (UT-Gel_H20 data line). After washing with SDS the fluorescent signal was close to control samples without gelatine. In contrast, PAC samples had much improved edge fidelity and after SDS washing showed a decrease in fluorescent intensity, but the printed lines (FIG. 6A) were still sharp and bright, indicating this retained gelatine was covalently bound to the surface.

### PAC Treatment of Coverslips Efficiently Promote Neural Differentiation of Mouse Embryonic Stem Cells.

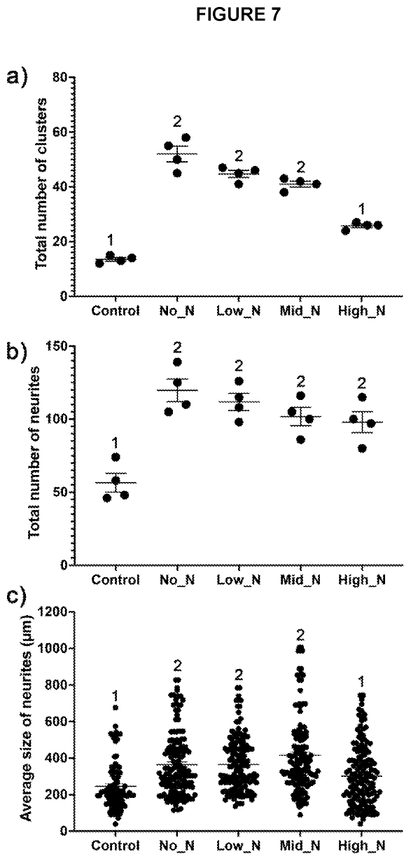

Mouse embryonic stem cells do not easily adhere to glass. Using PAC glass coverslips with different formulations of nitrogen and argon promotes mouse ES cell attachment. A greater attachment of cells was observed in all conditions compared to the untreated glass coverslip (FIG. 7).

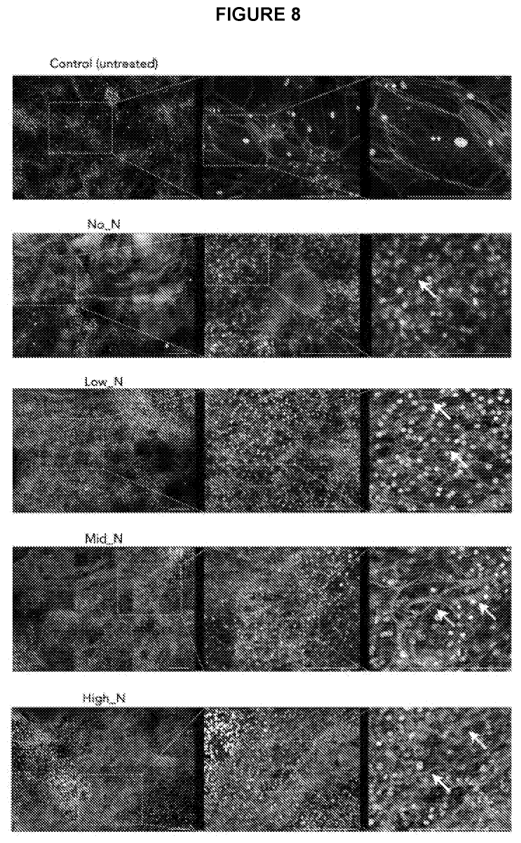

With the exception of the High_N coating, all PACs led to significantly greater numbers of neuronal clusters differentiating from mouse ES cells (FIG. 7A, P<0.05). Neuronal clusters were defined as an attached embryoid body with neurites stemming from the periphery of the body (FIG. 8). Additionally, the number of neurites projecting from the clusters were significantly greater in all PACs compared to the untreated control (FIG. 7B, P<0.05). The size of these neurites was significantly greater in all PACs except for the High_N coating (FIG. 7C, P<0.05). Thus, neuronal cell differentiation was efficiently achieved without the use of pre-attached proteins such as poly-l-lysine, gelatine or Matrigel.

To better understand the morphology of these neurites, confocal imaging was performed on the terminally differentiated neurons in all conditions (FIG. 8). Mouse ES cell-derived neurons were stained with Phalloidin to visualise the f-actin staining of the neurites (FIG. 8, magenta). Cells attached on the control coverslip were sparse, large and flat, resembling surface ectoderm differentiation (FIG. 8, **). Neurite coverage was expansive in all formulations of PAC glass coverslips (FIG. 8, arrowheads). No discernible differences in morphology were observed between the four different PACs by confocal microscopy.

Investigation of Protein Attachment to PACs from Defined Cell Culture Media Using Mass Spectroscopy.

1D Liquid chromatography mass spectrometry (LCMS) was used to determine the nature of the proteins as absorbed and those which were covalently attached to the PAC surfaces from the neural induction cell culture media to determine if the protein corona was different and how these differences related to the neural cell differentiation and surface radical density. Proteins were identified from the LCMS data using the MASCOT database, resulting in 91 unique proteins identified across all 10 conditions; the proteins per sample are summarized in Table 2. After filtering, a total of 22 unique proteins were characterised by their molecular weight, predicted isoelectric point, and net charge at pH 7.4.

Table 2: Total number of proteins and total peptide spectral matches (PSM) detected on all surfaces with and without SDS washing.

TABLE 2

| | |
|---|---|
| Mild wash | SDS wash |

| | Proteins | PSM | Proteins | PSM |
|---|---|---|---|---|
| Glass | 50 | 901 | 22 | 1142 |
| No_N | 39 | 309 | 36 | 1259 |
| Low_N | 46 | 645 | 34 | 878 |
| Med_N | 12 | 192 | 11 | 139 |
| High_N | 24 | 196 | 9 | 108 |

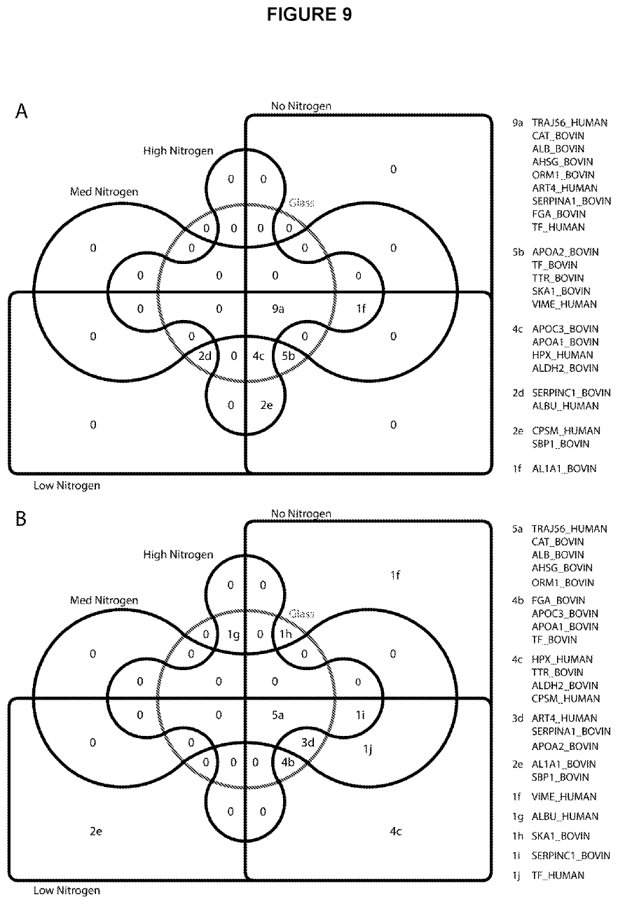

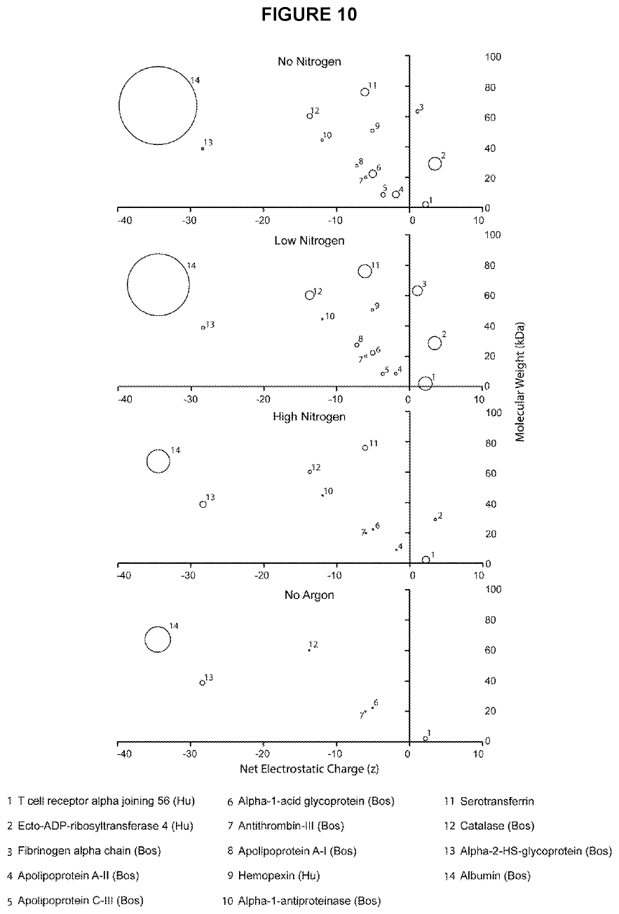

FIG. 9 shows the overlap of the 23 identified proteins across each of the PAC types compared with untreated glass without SDS washing (A) and with SDS washing (B). Post SDS washing of the 23 proteins, 7 were unique to either or both No_N and Low_N PAC, whereas there were no unique proteins on either Mid_N or High_N. The top 14 abundant proteins are characterised in FIG. 10 comparing the identified protein charge, molecular weight, and the total number of peptide spectral matches for each of the four PAC surfaces after washing with SDS. The level of nitrogen content in the PAC affected both the diversity of the protein layer and the total abundance of protein as inferred from Table 2. Of the most abundant proteins, No_N and Low_N PACs contained more positively charged proteins, which is likely due to these surfaces having a more negative surface charge relative to Mid_N and High_N PACs. The increased abundance of positively charged proteins correlated with increased diversity of small (<40 kDa) negatively charged proteins. As the nitrogen content decreased, larger, mildly negatively charged proteins were also found in higher abundance, notably catalase and serotransferrin. Only two large proteins with a charge <−25 m V were observed on all surfaces with albumin following the same inverse correlation with increasing nitrogen content. Alpha-2-HS-glycoprotein in contrast only showed a slight but opposing trend with regards to nitrogen content. However, as all characterised proteins observed aside from albumin, catalase, serotransferrin originated from the impurities in the B27 and N2 media, only general trends based on the size, charge and molecular weight are discussed.

The difference of cell attachment and differentiation in the experiments appears to be governed by the protein profiles at the substrate interface as found by mass spectroscopy (FIGS. 9 and 10). Influence factors of PAC to protein attachment include surface chemistry, charge and radical density. The immobilisation of proteins and other molecules from the culture media on a surface may occur in two steps: first, the molecules approach the surface by long-range and short-range forces and then they interact with the surface to optimize their surface energy status. The varieties of molecules approaching a surface may be determined by the surface charge of substrates and the molecules in the culture media. Based on the distribution of proteins profiles and their charges on PAC surfaces (FIG. 10), it is hypothesized that there is a relationship between the surface charge and protein diversity. In particular, PAC surfaces with low nitrogen content may be dominated by carboxylic groups and hence, tend to attract positively charged molecules. Small positive proteins are more dynamic and may be the first to arrive at the surface followed by progressive arrival of small negatively charged proteins and finally the large negatively charged proteins. On the contrary, PAC with high nitrogen content tends to have more positively charged groups. Very few small positively charged proteins were observed on the surface while mostly large and abundant negatively charged proteins in culture media were found on the surface. Although 13 of the 22 proteins were also found in abundance on untreated glass, the biological response of mouse ES cells to these proteins was measurably different (FIG. 6) to the same or similar proteins on No_N, Low_N and Mid_N PAC. The major difference is that those proteins are physiosorbed on glass surface while they are covalently immobilised on PAC surfaces. On a glass surface, the molecules temporarily adsorb to the surface and will be displaced by other molecules with higher surface affinity. On PAC surfaces, radicals emerging from the coatings may react with the adsorbed molecules to form covalent bonds and hence disable the displacement. Radical density likely plays an important role in this second step. This is evidenced by a higher number of proteins detected on No-N and Low-N surfaces after SDS wash (Table 2) which have higher radical density than others (FIG. 4B).

The results demonstrate the utility and robustness of the herein disclosed coated glass substrates for promoting cell attachment and efficient differentiation of neurites from undifferentiated mouse pluripotent stem cells. Prior art processes are complex, time-consuming and require toxic wet chemistry to link proteins onto the glass surface. The herein disclosed approach provides a safe and simple two-step process for the attachment and differentiation of pluripotent stem cells.

The differentiation of mouse EC into neuronal-like cells has been demonstrated as a simple process on plasma coated glass coverslips without the need for conventional prefunctionalized extracellular matrix strategies. The herein disclosed plasma-activated coated glass substrates comprise long-lived radicals which irreversibly attach biomolecules from cell culture media, forming a signalling interface to direct EC differentiation. PACs with low nitrogen content promote denser neurite clusters with larger size than PACs with high nitrogen content and bare glass. The surface chemistry, transparency and radical density of PACs may be controlled by changing the ratio of precursor gases introduced into plasma. Mass spectroscopy analysis showed different biomolecules profiles immobilized at the cell-surface interface, providing further information to explain the difference in cell culture.

### EXAMPLES

### Example 1: Plasma-Activated Coating (PAC) on Glass Coverslips

Glass coverslips (rectangle 22 cm×22 cm or round 12 mm in diameter, 170 μm thick) were purchased from Livingstone International and soaked and sonicated in ethanol before use. The surface plasma treatment of glass coverslips included two steps: surface activation and plasma deposition of a thin film coating. Cover slips were placed on a stainless-steel sample holder inside a plasma chamber which was then evacuated to a base pressure of 5×10⁻⁵ Torr. Plasma was generated using a capacitively coupled radio frequency generator (Eni OEM-6) with a power of 75 W for 10 minutes in 75 mTorr of argon. During this surface activation step, a pulsed bias of −500V, with a frequency of 3000 Hz and 20 μs pulse length, was applied to the sample holder by a RUP6 pulse generator (GBS Elektronik GmbH, Dresden, Germany). The chamber was again evacuated to the base pressure and then a mixture of gases including acetylene, nitrogen and argon with different ratios (see Table 1) was introduced into the chamber. The four gas mixtures were chosen with the same flow rate of acetylene and a constant total flow rate, while varying the nitrogen and argon flow rates. Pressure during deposition was kept constant at 110 mTorr. The deposition occurred for 10 minutes at 50 W radio frequency power while the same negative pulsed bias from RUP6 was applied. After plasma coating, samples were stored in a petri dish in ambient laboratory conditions for further analysis.

### Example 2: Surface Characterisation of PAC

Contact angles of two liquid probes (water and diiodomethane) were measured on the plasma coated and ethanol cleaned glass coverslips and polystyrene film (0.19 mm, Goodfellow Ltd, Cambridge) using a Theta tensiometer (ATA Scientific Instruments). The average of five contact angles of each liquid probe was used to calculate surface energy using Owens-Wendt-Rabel-Kaeble method. PAC samples were measured 1 day and 3 months after deposition of the coating and stored under ambient laboratory conditions during this time.

Surface chemistry of the coatings was investigated using transmission fourier transform infrared spectroscopy (T-FTIR) and X-ray photoelectron spectroscopy (XPS). For T-FTIR, PAC was deposited on high purity silicon wafers (B-doped p-type, 1-10 Ω·cm resistivity, 100 orientation) to obtain a good signal to noise ratio. FTIR spectra were collected one day after deposition with 500 scans with a resolution of 4 cm-1 in vacuum using a Vertex 70v spectrometer (Bruker). Spectra of PAC were obtained by subtracting the spectrum of the bare silicon wafer from the spectra obtained from PAC on the silicon wafer. For XPS analysis, PAC were deposited on thin oxide silicon wafers and analysed after storage for 1 week under laboratory ambient conditions. Survey scans (step size of 1 eV) and high-resolution scans of C1s, N1s, O1s and Si2p (step size of 0.1 eV) were recorded using a Thermo Scientific™ K-Alpha+™ spectrometer (ThermoFisher Scientific, UK). Areas of the peaks from high resolution scans were integrated to calculate the atomic percentage of elements on each surface. The C1s peaks were fitted with Lorentzian-Gaussian peaks using the following criteria: all deconvoluted peaks have the same full width half maximum and same ratio of Lorentzian/Gaussian mix, binding energy of C—C, C—O, C═O and O—C═O are approximately 1.5 eV apart respectively. The optical properties of PAC were characterized using spectroscopic ellipsometry and transmission spectroscopy. PAC coated silicon wafers (B-doped p-type, 1-100 Ω·cm resistivity, 100 orientation) were used for ellipsometry measurement (JA Woollam M2000 spectroscopic ellipsometer) with three angles (65, 70 and 75 degree). Psi and delta plots were fitted using a Cauchy layer to obtain coating thickness and refractive index. To study the transparency of PAC, PAC coated glass cover slips were scanned in the visible and UV range using a spectrophotometer (Cary, Agilent). An ethanol-cleaned glass cover slip was used as a control.

### Example 3: Radical Decay of PAC During Storage and Ability to Form Covalent Bonds with Extra Cellular Matrix Proteins

Radicals in PAC coatings were measured using electron spin resonance (EPR) (SpinScanX, Adani, Belarus). PAC was deposited on quartz slides (40×50×0.9 mm) which were fitted into the resonance cavity of the EPR equipment. Samples were measured at different time points during storage to investigate radical decay over time. The measurements were conducted at room temperature with a microwave frequency of 9.40 MHz and a central magnetic field of 336 mT with a sweep width of 30 mT and a microwave power of 0.946 mW. Origin Pro software was used to integrate the peaks to obtain peak areas which correlate to radical density. Microwave frequency and peak position were used to calculate the g-factor. Results reported are an average of three measurements at each time point.

To demonstrate the covalent attachment of an extracellular matrix at desired locations on PAC, microcontact printing of gelatine and a stringent wash with detergent was used to test the bond strength. Polydimethylsiloxane (PDMS) stamps were prepared by lithography with parallel stripes of 50 μm width with 100 μm gaps in between. They were treated with oxygen plasma (Harrick Plasma) for 3 minutes prior to use. Fluorescein conjugated gelatine from pig skin (Life Technology Australia) was prepared at a concentration of 0.1 mg/mL in PBS (phosphate buffered saline) buffer. A drop of 20 μL gelatine solution was placed on a PDMS stamp and incubated at room temperature. After 30 minutes, stamps were briefly rinsed with PBS and the excess liquid on the stamp surface was absorbed using Kim wipes. The stamps were placed onto the sample surfaces [UT (untreated glass) and PAC (plasma activated coating)] with a small weight support (˜30 g) to enhance the contact for 10 minutes. Samples were subsequently rinsed with milli Q water on a shaker (30 rpm) for 30 minutes to remove unbound molecules before drying for confocal fluorescence microscopy. A more stringent wash with 5% sodium dodecyl sulphate (SDS) was used to remove physisorbed molecules on the sample surface and to identify covalently bound molecules.

Confocal images were collected using Leica SP5 (Leica) microscope. Images were acquired using a 20× water immersion objective at either 1× magnification (low resolution) or 4× magnification (high resolution) with a 488 nm excitation laser.

### Example 4: Attachment and Differentiation of Mouse Embryonic Stem (ES) Cells on PAC Glass Coverslips

Wild-type, undifferentiated mouse embryonic stem (ES) cells (129SV/J, (Kuo, Zhou, Cosco, & Gitschier, 2001) were maintained in mouse ES cell maintenance media including Dulbecco's Modified Eagle Medium (DMEM) supplemented with 10% (v/v) foetal calf serum (FCS) (Bovogen), 2 mM Glutamax™ (ThermoFisher Scientific), 1 mM sodium pyruvate (ThermoFisher Scientific), 50,000 U·mL-1 penicillin, 50 mg·mL-1, streptomycin (ThermoFisher Scientific) and 150 μM of 1-thioglycerol (Sigma Aldrich). Leukaemia inhibitory factor (ProspecBio) was added at a concentration of 100 units·mL-1 to inhibit spontaneous mouse ES cell differentiation.

Mouse ES cell maintenance media was aspirated and the cells were washed with 1 mL PBS. After that, 500 μL of TrypLER Express was added to replace PBS and cells were incubated at 37° C. for 3 minutes to initiate dissociation. Afterwards, 1.5 mL of mouse ES cell maintenance media was added to quench the dissociation. The cell mixture was transferred into a 15 mL Corning tube and centrifuged at 240×g for 5 minutes. The supernatant was discarded and the cell pellet was resuspended in 1 mL of maintenance media.

To induce differentiation of mouse ES cells towards a neural lineage, 2.5×10⁴ cells were plated on PAC glass coverslips distributed across the four different recipes outlined above as well as control (untreated glass coverslips). Mouse ES cells were supplemented with neural induction media (Q.-L. Ying, M. Stavridis, D. Griffiths, M. Li, A. Smith, Nature Biotechnology 2003, 21, 183). Briefly, media consisted of 50:50 DMEM/F12: Neurobasal (ThermoFisher Scientific) media supplemented with 1× N2 (ThermoFisher Scientific), 1× B27 (ThermoFisher Scientific), 1 mM Glutamax™, 50,000 U·mL-1 penicillin, 50 mg·mL-1, streptomycin, 2.5 mM sodium ascorbate (Sigma Aldrich) and 450 μM of 1-thioglycerol. Media was changed every two days to promote cell survival until day 22 of differentiation. Total number of neuronal cell clusters, neurites and neurite length were measured using ImageJ and individual values plotted using GraphPad Prism (n=4, three independent experiments). Data comparing the size and number of neurites differentiated from mouse ES cells are presented as mean±SEM. Graphpad Prism 7 was used to generate graphs and determine statistical significance. A two-tailed one-way ANOVA with Tukey's posthoc analysis was used to compare the size and number of neurites differentiated between the untreated coverslips and PAC glass coverslip formulations. A p-value less than 0.05 was deemed significant. After 22 days, media was aspirated and differentiated mouse ES cells were washed twice with PBS. Following, differentiated cells were fixed with 4% paraformaldehyde (PFA) (in PBS) for 10-20 min at room temperature. Coverslips were then washed three times with PBS and once with PBS supplemented with 1% (v/v) Triton-X (PBST). Differentiated cells were then stained with 1 μM of Phalloidin (Sigma Aldrich) in PBST overnight at 4° C. The following day, Phalloidin-stained cells were washed three times with PBS and mounted in Vectashield containing DAPI (Vector Laboratories, USA). High resolution images were taken using a Zeiss LSM 800 confocal microscope coupled with the Zen Blue software package. Phalloidin was excited using the 565 nm laser while DAPI was excited using the 405 nm laser. Images were taken at 20× magnification. A 3×3 area focused on the centre of the coverslip was imaged and stitched together. Post-processing of images was performed on ImageJ (NIH) and data graphed using GraphPad Prism 7.

### Example 5: Mass Spectroscopy Analysis

PAC and UT glass coverslips were incubated in complete neural induction media (as above) for 1 hour at 37° C. in a cell culture incubator. Complete media was removed and the coverslips were washed in either PBS or 5% SDS for 1 hour at 37° C. on an orbital shaker. Coverslips were rinsed with PBS then moved to a clean 96 well plate and stored at −20° C. Coverslips were analysed by 1D liquid chromatography mass spectrometry (LC/MS). Coverslips were stored overnight at 4° C. prior to digestion in 12 ng/μL trypsin and 25 mM ammonium bicarbonate at 37° C. overnight. Samples were ZipTipped and eluted in 50% acetonitrile and 0.1% trifluoroacetic acid. The eluate was dried down and reconstituted in 5 μL loading buffer. Samples were subsequently diluted 1:10 and 3 μL was loaded onto LC/MS (QSTAR, Applied Biosystems). Acquired spectra were processed using the Mascot databases for human and bovine. Identified proteins with low peptide spectrum match (PSM) counts found on only one sample were excluded from further analysis. Additionally, human keratin hits were also excluded due to being an environmental contaminant.

### Example 6: Hydrogel Attachment on PAC Glass Coverslips

### Attachment of Hydrogel Layer

Methacrylated polyvinyl alcohol (PVA-MA) monomers were dissolved in milli-Q water in a 1 mL Eppendorf tube to form a 10 w/w % solution. The solution was vortexed for 1 minute and heated to 80° C. for 15 minutes to completely dissolve the monomers. The solution was then diluted into 0.1, 0.5 and 2 w/w % solutions. A volume of 500 μL PVA-MA solution was added on PAC coated glass coverslips (22×22 mm) prepared as in Example 1 for incubation (similar samples were prepared using quartz or silicon wafer substrates). The coverslips were placed in a petri dish and sealed using parafilm to prevent evaporation. The incubation was conducted in an oven at 30° C. for 3 hours in an atmosphere of argon. After incubation, samples were rinsed twice in milli-Q water. The petri dish was then filled with milli-Q water, sealed with parafilm and placed on an orbital shaker at 100 rpm overnight to remove unbound molecules. The samples were dried in a vacuum oven at 37° C. for 2.5 hours before analysing using Ellipsometry to confirm the forming of hydrogel layer.

### Radical Measurement

EPR measurements were conducted using SpinScanX equipment (ADANI). Quartz slides (Helm Australia) of 5×40 mm were used as substrates which do not contain free radicals. Quartz slides had a PAC coating formed from 1:3:10 (acetylene:nitrogen:argon) deposited on them. EPR measurements were taken at room temperature, with a microwave frequency of 9.4 GHz, central magnetic field of 336 mT, sweep width of 15 mT, and microwave power of 0.947 mW. Five scans were taken, and EPR measurements given as the average value of these scans. An initial measurement was conducted immediately after removal of the coated sample from the PAC chamber. One slide was then incubated in 800 UL of milli-Q water, and the other in 800 μL of 1 w/w % PVA-MA solution (milli-Q solvent). Both slides were incubated at 30° C. for 3 hours. After incubation, they were rinsed twice in milli-Q water, and dried under vacuum for 1 hour. A second EPR measurement was taken after completion of drying.

### Formation of Additional Hydrogel Layer on Top of the Radical-Induced Hydrogel Attachment

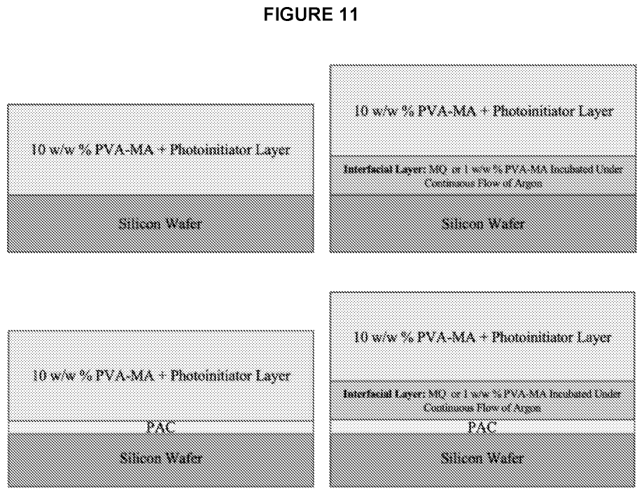

A 10 w/w % PVA-MA with photoinitiator solution was pipetted onto the samples having hydrogel attached to the PAC coated substrates. To test the effect the intermediate layer (of hydrogel prepared under argon incubation) had on adhesive strength of the polymerisation to the PAC surface, a second group of samples which had no additional preparation after PAC treatment were tested. The 10 w/w % PVA-MA with photoinitiator solution was then pipetted directly onto the samples, which were either PAC or untreated substrate surfaces. Volumes were varied between experiments to determine volumes which yielded the best hydrogel geometry and thickness, but were between 75 and 250 μL. The photoinitiator solution was left on the surface for 10 minutes. Samples were then irradiated with 365 nm UV light for 10 minutes. The photoinitiator solution was left on the samples overnight, and washed off using milli-Q water the next day. Washing was performed by rinsing samples twice under continuous flow of milli-Q water. After washing, samples were placed in a vacuum oven at 37° C. for 24 hours. Samples were then submerged in milli-Q water and allowed to reswell for 25 minutes. After re-swelling, the adhesion of hydrogel on the sample surface was examined by applying force to the gel, either shaking the sample in a water bath or poking the samples with a pair of tweezers. Observations as to the deformation and damage inflicted on the gel after applying this force were made. FIG. 11 illustrates the four different layered materials that were prepared on a silicon substrate.

### Adhesion Tests

To evaluate the adhesion of hydrogel on PAC coated silicon wafer, adhesion tests were performed using an Instron Mechanical Tester. An adhesive tape was applied on the sample surface, covered the hydrogel area. A tensile force was used to peel this tape off the surface. If the adhesion between the hydrogel and the PAC surface was weaker than the adhesion between the tape and hydrogel, the hydrogel was peeled off from the PAC surface. If the breaks occurred at the tape, then the measured force represented a lower bound for the force required to delaminate the layers.

To quantify the adhesion strength, tape tests were performed, allowing the force required to either delaminate the hydrogel from the PAC, or tear the PVA-MA hydrogel, to be determined. It was observed that the hydrogel layer remained on a glass surface after pulling the adhesive tape, making the exact adhesive strength impossible to be calculated. However, hydrogel structures retained their size and shape, and displayed no obvious signs of tearing.

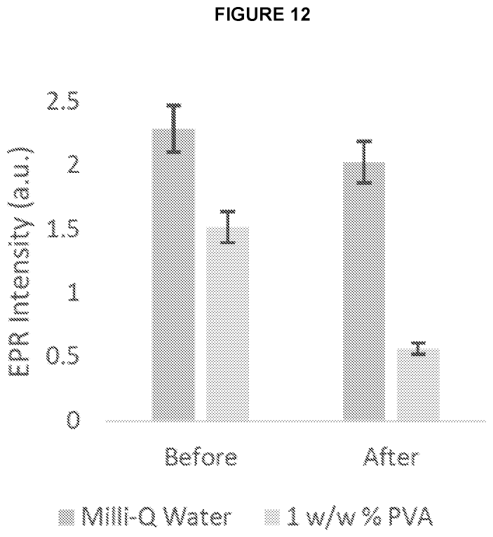

A very thin film of hydrogel (less than 1 nm) can be detected on PAC and untreated glass and silicon wafers using Ellipsometry Spectroscopy but as this thickness had reached the limit of detection of this technique it was difficult to compare the difference in thickness of hydrogel formation on both surfaces. However, the EPR measurement on PAC before and after PVA-MA incubation (FIG. 12) shows a significant reduction of radical content, compared to the control in milli-Q water, indicating that there are reactions between radicals in PAC and monomers. The reactions form covalent bonds of hydrogel layer on PAC which are stronger than the physical adsorption on bare glass or silicon wafers. This strong attachment was evidenced from the growth of additional hydrogel forming on top of this layer using photo initiator and the dehydration-reswelling test. Visible hydrogels formed on all samples described in FIG. 12. Drying and re-wetting of hydrogel were performed, with the expectation that complete drying of the structure followed by re-wetting and swelling of the hydrogel would stress and break weak bonds between the hydrogel and the surface. It was observed that hydrogels on untreated silicon wafer (FIG. 12-a and b) were easily removed with minimal force, either by shaking the sample inside a water bath or lightly poking with a tweezer, resulting in the hydrogel lifting off the surface in one piece. The 10 w/w % PVA-MA+Photoinitiator on PAC samples (FIG. 12-c) also had weak adhesion of the hydrogel to the sample surface. Scratching upon the application of the deformation force was only observed for one out of five repeats, while samples lifted straight off for the other four repeats. Only the hydrogels of 10 w/w % PVA-MA+Photoinitiator on Interfacial layer sample (FIG. 12-d) was unable to be completely removed from the sample surfaces. On three of five repeats, poking these samples with a tweezer resulted in the hydrogel being scratched. On the two remaining repeats, the hydrogel structures were only partially able to be removed, with the force required to scratch the surface greater than for all other treatment groups.

Without wishing to be bound by theory, it is likely that the polymerization of hydrogel monomers via the photoinitiator occurred faster than the covalent attachment of hydrogel monomers to the PAC surface, therefore, it is an already formed hydrogel layer that attaches to the PAC surface in the structure in FIG. 12(d), rather than the growth of polymer chains from PAC blending into the hydrogel structure. Further, as the formed hydrogel is hydroscopic it may alter the bulk interactions at the PAC-water interface possibly pulling any free monomer or free reactive side chains away from the PAC surface and into the growing hydrogel mesh.

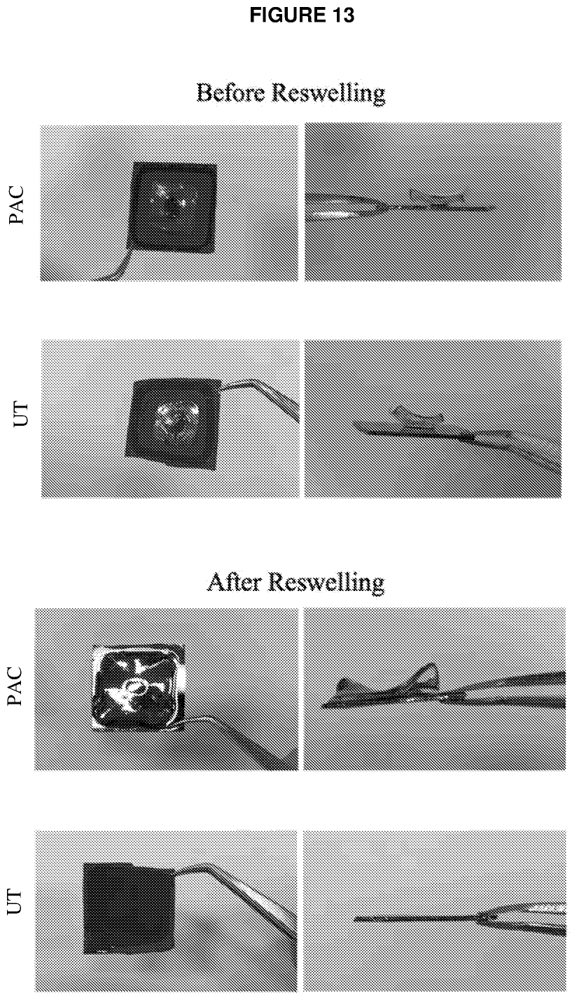

FIG. 13 compares the hydrogel on PAC and untreated silicon wafer, showing the attachment of hydrogel on PAC, while it was completely removed from the surface of bare silicon (UT) after reswelling.

### Example 7: Stability of PAC in Simulated Body Fluid

The stability of PAC adhesion on glass is crucial for successful cell culture, which typically occurs in culture media over several weeks. To assess the stability of the coating in simulated body fluid (SBF) for a month, a test was conducted by comparing the surface composition of samples before and after a 4-week soak in SBF. Glass slide cuttings (2.5 cm×1.5 cm) were utilised as substrates for plasma coatings with four PAC recipes, as set out in Table 1. The samples were stored in a 6-well plate at ambient conditions for one week before the experiment. To prevent contamination, the SBF was prepared and filtered using a 0.22-μm microfilter, and the samples treated with UV light (10 minutes each side) in a safety cabinet before adding SBF (4 ml/well). The well plate was then wrapped in parafilm and placed in a 37° C. incubator for a month. To prevent concentration changes, the SBF was replaced every week.

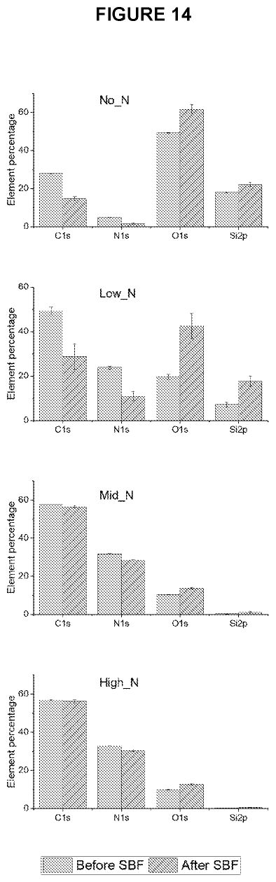

The average of XPS analysis of three locations on each sample was compared before and after the incubation to investigate the presence of the coating. A significant reduction of nitrogen and carbon and an increase of silicon are indications of coating loss. Results are presented in FIG. 14 and it is evident that little carbon or nitrogen were lost from the Mid_N and High_N samples.

### Example 8: Laminin Immobilisation

### Deposition of Plasma Activated Coating (PAC)

Plasma activated coating was deposited onto silicon wafers and glass coverslips in a two-step process similar to that of Example 1. To initiate the process, the plasma chamber was first evacuated to a base pressure of 5×10⁻⁵ Torr and argon gas was introduced into the system at a constant mass flow rate of 37 standard cubic centimetres per minute (sccm). The plasma treatment was conducted for 5 minutes to activate the sample surface, with an RF power of 75 W (delivered by a capacitively coupled radio frequency generator Eni OEM-6) and a negative pulsed bias connected to the sample holder at 500V, 3 kHz and 20 μs. This negative pulsed bias was supplied by a RUP3 DC power supply connected to a pulse generator.

Subsequently, a reactive gaseous mixture of acetylene, nitrogen and argon was introduced in the system at constant mass flow rates of 1, 3, and 10 (for Low Nitrogen PAC) or 1, 10 and 3 (for Mid Nitrogen PAC) sccm respectively for plasma deposition. The plasma was generated by delivering and coupling the RF power of 50 W. During the plasma coating process, the same negative pulsed bias was applied to the sample holder. The samples were exposed to the reactive plasma for 10 min, leading to the deposition of approximately 9 nm (Low Nitrogen) or 25 nm (Mid Nitrogen) thick PAC layers.

### Optimisation Studies on the Deposition of PAC on Silicon Wafer

Etching the silicon wafer surface with argon plasma prior to plasma deposition provides optimal conditions for PAC adhesion to the substrate. Therefore, it was of interest to examine this step in more detail to optimise retention of the PAC coating.

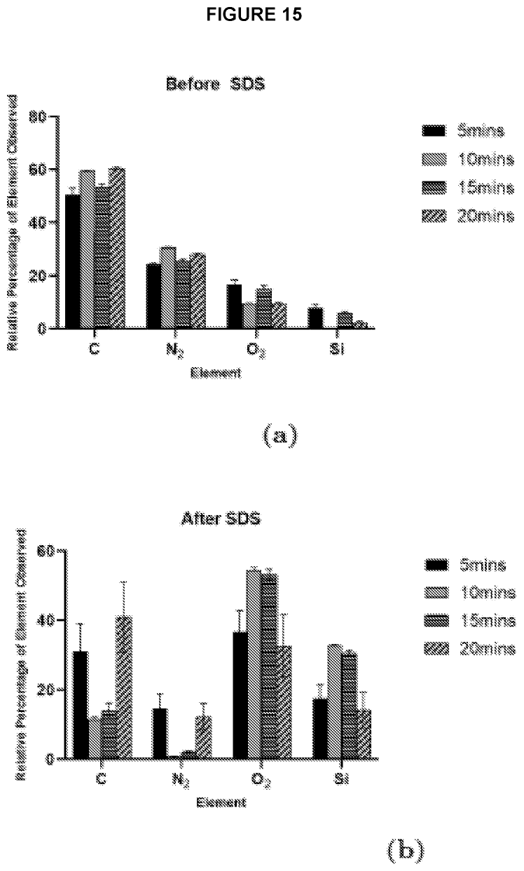

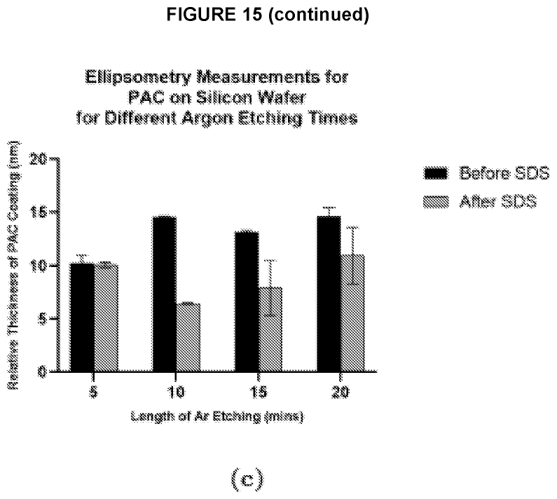

XPS measurements before and after sodium dodecyl sulphate (SDS) washing revealed differences in the surface of the PAC layer on the silicon wafers. It indicated that the magnitude of the delamination of the PAC was lower when samples were treated with either 5 or 20 minutes of argon plasma prior to plasma deposition (FIGS. 15(a) and 15(b))

At these time points, the relative percentages of carbon and nitrogen remained significantly higher after rigorous washing with SDS than those samples with 10 or 15 minute argon treatment times. Conversely, the relative percentages of oxygen and silicon were lower after 5 or 20 minutes argon treatment than their counterparts at 10 and 15 minutes. The unidirectional measurement procedure of XPS means the PAC layer will be detected first before the silicon wafer, as well as the intermediate silicon dioxide layer, and therefore the samples where the percentage of silicon is much lower indicates a thick PAC coating is present, as is explained by the higher carbon and nitrogen values of which the plasma is mainly comprised. The opposite is true for those samples where the silicon and oxygen content has increased but carbon and nitrogen content has decreased, as this demonstrates a reduction in the PAC layer, exposing more of the silicon/silicon dioxide layers.

FIG. 15(c) presents further analysis of the samples using ellipsometry. This shows that there were varying thicknesses observed in the layers on each sample apart from those etched with argon plasma for only 5 minutes. After washing with SDS, samples etched for 10, 15 and 20 minutes either displayed a significant reduction in the amount of plasma attached to the surface (evidenced by the samples etched for 10 minutes) of the silicon, or displayed large variability in surface thickness, even amongst individual silicon wafer samples (as seen with the samples etched for 15 or 20 minutes). Conversely, those samples etched for 5 minutes with argon plasma displayed much higher consistency in surface thickness and did not lose PAC layer, as supported by the XPS data. Accordingly, a 5 minute argon etching time appeared preferred.

### Laminin Immobilisation on Silicon Wafer

Silicon wafer (Topsil) with or without plasma activated coating was cut using a diamond-tipped cutter into 1.2 mm² sample sizes and cleaned using a sonicator by immersing in falcon tubes containing 100% ethanol twice, followed by immersion in falcon tubes containing milliQ water. Samples were then dried overnight in a desiccator.

Laminin-521 (LN521) (Stemcell) was diluted to a working concentration of 10 μg/mL in phosphate buffered saline (PBS) containing Ca2+ and Mg2+ (Gibco), prepared according to the manufacturer's instructions, and deposited onto the silicon wafer for 3 hours at 37° C. The samples were then washed 3 times with PBS containing Ca2+ and Mg2+, and once with milliQ water to remove any residual salts. For SDS washing, dodecyl sulfate sodium salt (EMD Millipore) was diluted to a 2% concentration in milliQ water. Si wafer samples were placed in individual falcon tubes containing this solution and heated using a water bath at 70° C. for 1 hour. Samples were then washed 3 times with milliQ water and dried in a vacuum oven overnight before being sealed in a 12-well plate for surface analysis.

### X-Ray Photoelectron Spectroscopy

The surface chemical composition of the PAC treated silicon wafers was investigated using XPS with a Thermo Scientific™ K-Alpha™ spectrometer (ThermoFisher Scientific, UK). For these measurements, silicon wafers (12 mm×12 mm) were washed with ethanol and treated with PAC as described above, followed by incubation with Laminin, as described above. The samples were then mounted on the XPS sample holder using conductive carbon tape. Survey spectra of the samples were acquired with a step size of 1 eV while high resolution spectra of C1s, N1s, O1s, Si2p and S2p were collected with a step size of 0.1 eV. Elemental composition of the coating and proteins was calculated by integrating the high resolution spectra using Advantage software (version 5.9902).

### Spectroscopic Ellipsometry

Ellipsometry was used to provide complementary information about Laminin attachment and coverage. The characterisation was conducted using a J. A. Woollam M-2000 spectroscopic ellipsometer for the visible wavelength range. Silicon wafers were used in place of glass coverslips so as to improve the reflection signal. Samples (12 mm×12 mm) were washed with ethanol and treated with PAC as described above, followed by incubation with Laminin as is described above, prior to being placed in the spectroscopic ellipsometer for measurement. Data was collected at three angles of incidence (65°, 70°, and 75°) before plasma coating, after plasma coating and then again after Laminin attachment. A model was fitted for each data set with the unknown parameters restricted to the topmost layer. The parameters used for the previous layers were imported from models fitted to the preceding data sets.

Cell Culture—iPSC Culture

Human induced pluripotent stem cells (iPSCs) (Thermo Fisher) were cultured on matrigel in a 6-well plate (Corning Costar) in mTeSR plus medium with supplement added. All reagents and protocol were from StemCell Technologies. Briefly, mTeSR media was changed the following day to dilute out ROCK inhibitor, then changed subsequently every two days after. Upon reaching a confluency of 80%, cells were passaged into a new 6-well plate using ReLeSR and fresh mTeSR containing ROCK inhibitor, while any other cells not required for passaging were frozen down using CryoStar freezing medium, or plated for differentiation experiments.

Cell Culture—iPSC Differentiation

Undifferentiated human iPSCs were cultured as above. Upon passaging, they were plated in a 12-well plate (Corning Costar) at a density of approximately 1.58×10⁵ cells/cm2 (6×10⁵ cells/well). The reagents and protocol used were from StemCell Technologies. Briefly, the day after seeding, (day −1) fresh mTeSR medium with supplement was added to dilute out the ROCK inhibitor. The following day (day 0), CM differentiation media A was added (with 10 μg/mL matrigel introduced topically) for two days. Differentiation media B was added on day 2, followed by differentiation media C on days 4 and 6. Finally, the differentiation media was replaced with CM maintenance media on day 8 and was replenished every 2 days.

### Covalent Attachment of Laminin-521

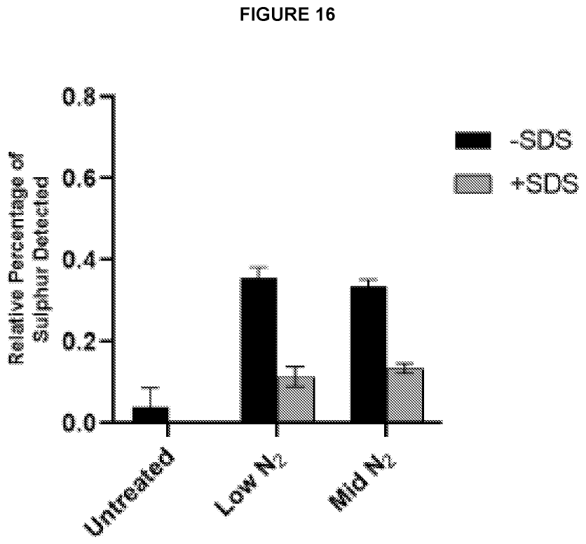

Plasma treated surfaces have excellent promise to covalently immobilise LN521. XPS analysis for sulphur (present in LN521 from the abundance of disulphide bridges in the molecule) on the silicon wafer surfaces shows that the plasma-coated samples sustained a higher quantity of LN521 on the surface to begin with (FIG. 16).

When washed with SDS to remove any non-covalently bound molecules, a substantial proportion of LN521 remained on the surface indicating the molecule was successfully bound covalently. Moreover, untreated silicon wafer displayed an inherently lower binding capability prior to SDS washing and after removal of physisorbed molecules with the surfactant, no LN521 remained covalently attached.

iPSC-CM Differentiation

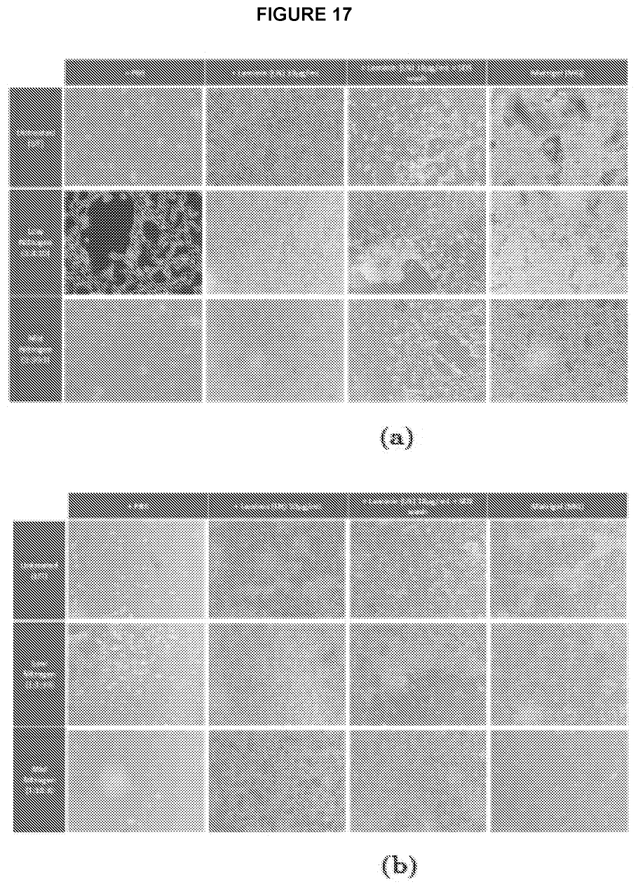

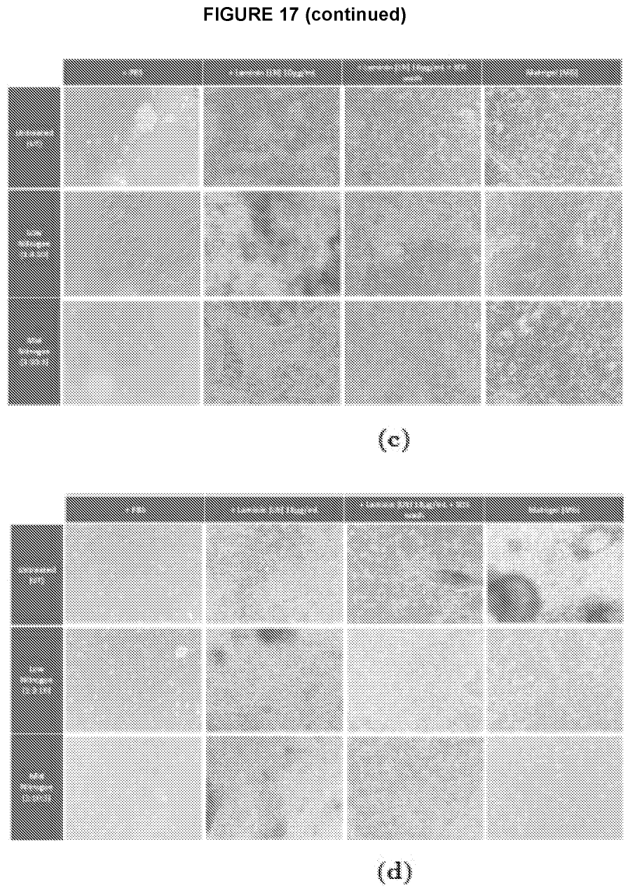

Human Induced Pluripotent Stem Cells were seeded into a 12-well plate as described above. During several stages of the differentiation process, brightfield images were taken to monitor the progress of the cells. FIG. 17 presents some of these findings at certain stages along the differentiation process.

Upon changing from differentiation media A to differentiation media B (17a), media C (17b), 2 days into the maintenance media (17c), and over a week into the maintenance media as the cells begin to develop into more mature CM (5d).

These results show that LN521 plays a crucial role in supporting the stem cell differentiation into CM, providing suitable signals and cues, and promoting an environment which aids in correctly directing the cells towards a cardiac lineage. This is evidenced by the rapid loss in cell viability from both the PBS controls (no Laminin) and the Matrigel controls, the current gold standard substrate used for iPSC culture.

By day 4, iPSCs on Laminin were beginning to transform along the mesoderm lineage, witnessed in part by the changes in morphology from a rounded shape to elongation, signifying differentiation. By day 10, cells had formed cardiac fibrils and beating was observed in several places (first signs of beating began by day 7). By day 17, individual colonies had coalesced to form a uniform sheet of beating CM which had begun to display a singular rhythmic beating pattern.

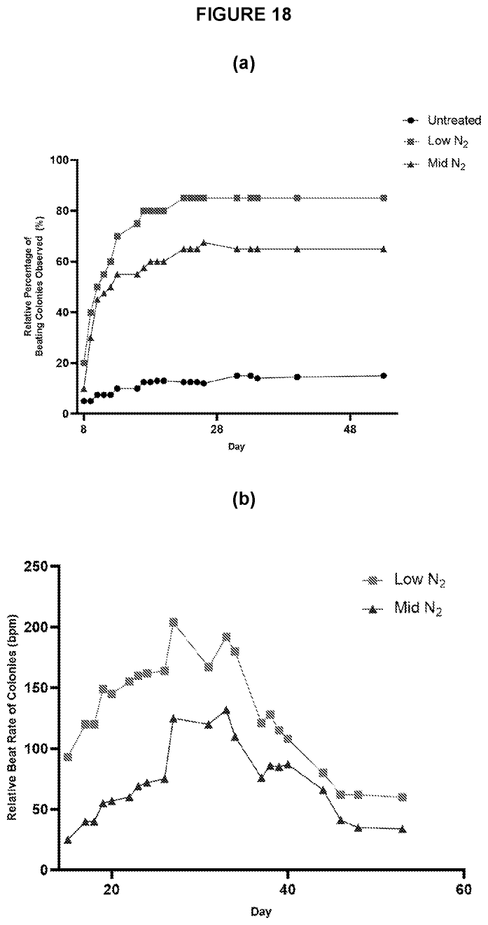

The advantage provided by the PAC in supporting cardiac differentiation and development is further highlighted in FIG. 18. Surfaces with immobilised LN521 and treated with low and mid nitrogen plasma recipes produced a significantly higher proportion of beating colonies compared to the untreated surface with LN521 (FIG. 18a). The covalent attachment of LN521 to the plasma-treated surfaces provided a consistent cardiac environment for the cells without the need to replenish LN521 to support cell viability. On the other hand, untreated samples struggled to produce a high yield of beating colonies due to the significant removal of Laminin from prior washing as well as during each media change which depleted the functionality of the surface. Furthermore, the high yield in cardiomyocyte colonies from samples with plasma treatment+LN521 permitted the formation of uniform sheets of beating cells. This allowed for a semi-quantitative analysis of the beating rate in both cases to be made (FIG. 18b). As the number of colonies coalescing increased and the cells began to mature, the beat rate in both cases escalated. By approximately day 30, the beating reached a plateau and began to slow down to a more stable rate.

Of notable significance is that those cells grown on low nitrogen plasma-treated coverslips experienced a higher beat rate as well as a faster development of beating CM, whereas those on the mid nitrogen plasma-treated coverslips produced a similarly-yet-lower yield of beating CM, but produced a lower heart rate more akin to an adult heartbeat, suggesting these cells might display a more adult behaviour and prove more useful in future scenarios concerning cardiac drug development assays.

1. A coated glass, quartz or silicon substrate, wherein the coating comprises a plasma-activated coating, said plasma-activated coating comprising radicals, and wherein the coated substrate is characterised by one or more of the following features:
a) a plasma-activated coating thickness from about 1 nm to about 100 nm;
b) a UV/visible absorbance in a range of about 320 nm to about 600 nm which is substantially the same as that of the substrate absent the plasma-activated coating;
c) an electron spin resonance peak centred at an applied magnetic field strength in a range of about 333 mT to about 339 mT;
d) electron spin resonance peaks centred at an applied magnetic field strength in a range from about 327 mT to about 332 mT and in a range from about 340 mT to about 345 mT;
e) a water contact angle of less than 60° measured within 1 day of preparation of the coated substrate, wherein the coated substrate is stored at ambient conditions prior to measurement;
f) a water contact angle of less than 80° measured within three months of preparation of the coated substrate, wherein the coated substrate is stored at ambient conditions prior to measurement;
g) a nitrogen:carbon elemental ratio of about 0.01:1 to about 1:1
h) infrared absorption peaks in the range 1400-1800 cm-1; and
i) an inability to remove all plasma-activated coating from the substrate with rigorous washing.

2. The coated substrate according to claim 1, wherein the plasma-activated coating thickness is from about 1 nm to about 100 nm, or from about 2 nm to about 50 nm, or from about 5 nm to about 40 nm.

3. The coated substrate according to claim 1 or claim 2, wherein the water contact angle after storage for 1 day under ambient conditions is less than 50°.

4. The coated substrate according to any one of claims 1 to 3, wherein the water contact angle after storage for three months under ambient conditions is less than 70°.

5. The coated substrate according to any one of claims 1 to 4, wherein the radical content of the plasma-activated coating decreases by less than 80% after storage for three months under ambient conditions.

6. The coated substrate according to any one of claims 1 to 5, wherein the nitrogen:carbon elemental ratio in the plasma-activated coating is from about 0.1:1 to about 2:3.

7. The coated substrate according to any one of claims 1 to 6, wherein the percentage elemental nitrogen as measured by XPS in the plasma-activated coating is from about 50% to about 1%, or from about 40% to about 1%.

8. The coated substrate according to any one of claims 1 to 7, wherein the percentage elemental carbon as measured by XPS in the plasma-activated coating is from about 90% to about 10%, or from about 70% to about 10%.

9. The coated substrate according to any one of claims 1 to 8, wherein the substrate comprises silicate glass, borosilicate glass, soda-lime glass or silicon oxide.

10. The coated glass substrate according to claim 9, wherein the glass substrate is a glass coverslip, a flat-bottomed glass dish or a glass cell culture chamber slide.

11. The coated substrate according to any one of claims 1 to 10, further comprising one or more hydrogels covalently bonded to the plasma activated coating.

12. The coated substrate according to claim 11, wherein the hydrogel is crosslinkable via radical initiation.

13. The coated substrate according to claim 11 or claim 12, wherein the hydrogel is peptide or protein based.

14. The coated substrate according to any one of claims 11 to 13, wherein the hydrogel comprises one or more of acrylated polyvinyl alcohol, such as, for example, methacrylated polyvinyl alcohol, GelMA (gelatin+methacrylic anhydride), polyacrylamide, silk hydrogel, hyaluronic acid hydrogels, and beta-peptide hydrogels modified with cell adhesive sequences, such as RGD (arginylglycylaspartic acid).

15. The coated substrate according to any one of claims 11 to 14, wherein the hydrogel comprises at least two layers, wherein a first layer of hydrogel is derived from reacting hydrogel monomers with the plasma-activated coating and a second layer of hydrogel is derived from polymerising hydrogel monomers, said second layer of hydrogel being disposed atop the first layer of hydrogel.

16. The coated substrate according to any one of claims 1 to 15, further comprising one or more biomolecules covalently bonded to the plasma-activated coating and/or hydrogel.

17. The coated substrate according to claim 16, wherein the one or more biomolecules comprise one or more proteins, polysaccharides, nucleotides, oligonucleotides, antioxidants, growth factors, vitamins and lipids.

18. The coated substrate according to claim 17, wherein the one or more proteins comprise one or more glycoproteins.

19. The coated substrate according to claim 18, wherein the glycoprotein comprises laminin.

20. The coated substrate according to any one of claims 1 to 19, further comprising extracellular matrix covalently bonded to the plasma-activated coating and/or hydrogel.

21. The coated substrate according to any one of claims 1 to 20, further comprising cells, wherein the cells are covalently bonded to or immobilised on the plasma-activated coating and/or hydrogel.

22. A process for producing a coated substrate according to any one of claims 1 to 10, comprising:
a) activating one or more surfaces of a substrate by exposing said substrate to a plasma formed in the presence of one or more of helium, neon, argon and xenon; and
b) depositing a plasma-activated coating on the one or more activated surfaces of the substrate by exposing said activated surface to a plasma formed in the presence of one or more organic gases, and one or more of nitrogen, helium, neon, argon and xenon;
wherein the total pressure in step b) is from about 25 mTorr (3.3 Pa) to about 500 mTorr (66.7 Pa).

23. The process according to claim 22, wherein the total pressure in step a) is from about 25 mTorr (3.3 Pa) to about 500 mTorr (66.7 Pa).

24. The process according to claim 22 or claim 23, wherein the total pressure in step b) is from about 25 mTorr (3.3 Pa) to about 350 mTorr (46.7 Pa), or from about 25 mTorr (3.3 Pa) to about 250 mTorr (33.3 Pa), or from about 25 mTorr (3.3 Pa) to about 150 mTorr (20.1 Pa).

25. The process according to claim 22 or claim 23, wherein the total pressure in step b) is less than about 250 mTorr (33.3 Pa), or less than about 200 mTorr (26.7 Pa).

26. The process according to any one of claims 22 to 25, wherein a plasma discharge in step a) is maintained from about 10 second to about 30 minutes, or from about 2 minutes to about 20 minutes.

27. The process according to any one of claims 22 to 26, wherein a plasma discharge in step b) is maintained from about 30 seconds to about 30 minutes, or from about 2 minutes to about 20 minutes.

28. The process according to any one of claims 22 to 27, wherein the plasma in step a) is formed in the presence of argon.

29. The process according to any one of claims 22 to 28, wherein the plasma in step b) is formed in the presence of nitrogen and one or more of helium, neon, argon and xenon.

30. The process according to any one of claims 22 to 29, wherein the plasma in step b) is formed in the presence of nitrogen and argon.

31. The process according to any one of claims 22 to 30, wherein the one or more organic gases comprises one or more of hydrocarbon and substituted hydrocarbon.

32. The process according to claim 31, wherein the substituted hydrocarbon comprises one or more of hydroxyl substituted hydrocarbon, amino substituted hydrocarbon, and hydrocarbons substituted with sulphur-containing groups.

33. The process according to any one of claims 22 to 31, wherein the one or more organic gases comprises one or more linear or branched alkane or cycloalkane, linear or branched alkene or cycloalkene, and linear or branched alkyne.

34. The process according to any one of claim 22 to 31 or 33, wherein the one or more organic gases comprises acetylene or substituted acetylene.

35. The process according to any one of claims 22 to 34, wherein the power supplied in step a) and step b) is in the form of DC power, pulsed DC power, or AC power, such as RF power or microwave power.

36. The process according to any one of claims 22 to 35, wherein the power supplied in step a) is from about 10 W to about 200 W, or from about 50 W to about 100 W.

37. The process according to any one of claims 22 to 36, wherein the power supplied in step b) is from about 10 W to about 200 W, or from about 25 W to about 100 W.

38. The process according to any one of claims 22 to 37, wherein during step (a), a pulsed bias of from about −200 V to about −1000 V, with a frequency from about 500 Hz to about 5000 Hz, and a pulse length from about 5 μs to about 20 μs, is applied.

39. The process according to any one of claims 22 to 38, wherein during step (b), a pulsed bias of from about −200 V to about −1000 V, with a frequency from about 500 Hz to about 5000 Hz, and a pulse length from about 5 μs to about 20 μs, is applied.

40. The process according to any one of claims 22 to 39, wherein the volume ratio of the at least one organic gas fed to the plasma chamber to the sum of one or more of nitrogen, helium, neon, argon and xenon, is about 1 to 20 to about 1 to 2, or from about 1 to 15 to about 1 to 5.

41. The process according to any one of claims 22 to 40, wherein the volume ratio of the at least one organic gas fed to the plasma chamber to the sum of nitrogen and argon is about 1 to 20 to about 1 to 2, or from about 1 to 15 to about 1 to 5.

42. The process for producing a coated substrate according to any one of claims 11 to 14, comprising the step of contacting one or more hydrogel monomers with the coated substrate according to any one of claims 1 to 10.

43. The process for producing a coated substrate according to claim 15, comprising the step of contacting one or more hydrogel monomers and initiator with the coated substrate according to any one of claims 11 to 14.

44. The process for producing a coated substrate according to any one of claims 16 to 21, comprising the step of contacting one or more biomolecules with the coated substrate according to any one of claims 1 to 15.

45. A method of cell differentiation comprising:
(a) depositing pluripotent cells in a cell culture medium on a plasma-activated coated substrate, or a plasma-activated coated substrate further comprising hydrogel, according to any one of claims 1 to 15;
(b) covalently attaching biomolecules from the cell culture medium to the plasma-activated coating and/or hydrogel; and
(c) allowing the cells to differentiate;
wherein the differentiation is directed by the covalently attached biomolecules and/or hydrogel.

46. A method of cell differentiation comprising:
(a) depositing biomolecules on a plasma-activated coated substrate, or a plasma-activated coated substrate further comprising hydrogel, according to any one of claims 1 to 15;
(b) covalently attaching one or more of the biomolecules to the plasma-activated coating and/or hydrogel;
(c) depositing pluripotent cells in a cell culture medium on the plasma-activated coating and/or hydrogel; and
(d) allowing the cells to differentiate;
wherein the differentiation is directed by the covalently attached biomolecules and/or hydrogels.

47. A method according to claim 45 or claim 46, wherein the method additionally improves cell attachment to a substrate and/or improves cell survival, improves cell proliferation and/or improves cell function.

48. A method of attaching cells to a substrate comprising:
(a) depositing cells in a cell culture medium on a plasma-activated coated substrate, or a plasma-activated coated substrate further comprising hydrogel, according to any one of claims 1 to 15;
(b) covalently attaching biomolecules from the cell culture medium to the plasma-activated coating and/or hydrogel; and
(c) attaching the cells to the covalently attached biomolecules.

49. A method of attaching cells to a substrate comprising:
(a) depositing biomolecules on a plasma-activated coated substrate, or a plasma-activated coated substrate further comprising hydrogel, according to any one of claims 1 to 15;
(b) covalently attaching one or more of the biomolecules to the plasma-activated coating and/or hydrogel;
(c) depositing cells in a cell culture medium on the plasma-activated coating and/or hydrogel; and
(d) attaching the cells to the covalently attached biomolecules.

50. A method of increasing cell survival on a substrate comprising:
(a) depositing cells in a cell culture medium on a plasma-activated coated substrate, or a plasma-activated coated substrate further comprising hydrogel, according to any one of claims 1 to 15;
(b) covalently attaching biomolecules from the cell culture medium to the plasma-activated coating and/or hydrogel; and
(c) attaching the cells to the covalently attached biomolecules.

51. A method of increasing cell survival on a substrate comprising:
(a) depositing biomolecules on a plasma-activated coated substrate, or a plasma-activated coated substrate further comprising hydrogel, according to any one of claims 1 to 15;
(b) covalently attaching one or more of the biomolecules to the plasma-activated coating and/or hydrogel;
(c) depositing cells in a cell culture medium on the plasma-activated coating and/or hydrogel; and
(d) attaching the cells to the covalently attached biomolecules.

52. The method according to any one of claims 45 to 51, wherein the biomolecules comprise one or more proteins, polysaccharides, nucleotides, oligonucleotides, antioxidants, growth factors, vitamins and lipids.

53. The method according to claim 52, wherein the one or more proteins comprise one or more glycoproteins.

54. The method according to claim 53, wherein the glycoprotein comprises laminin.
